(19) United States
(12) Patent Application Publication
(10) Pub. No.: US 2010/0197688 A1
(43) Pub. Date: Aug. 5, 2010

(54) EPHA4 RTK INHIBITORS FOR TREATMENT OF NEUROLOGICAL AND NEURODEGENERATIVE DISORDERS AND CANCER

(76) Inventors: Philippe G. Nantermet, Lansdale, PA (US); Hamaka A. Rajapakse, Wyncote, PA (US); Takeshi Sagara, Ibaraki (JP); John M. Sanders, Collegeville, PA (US); Hong Zhu, Lansdale, PA (US)

Correspondence Address: MERCK, P O BOX 2000, RAHWAY, NJ, 07065-0907, US

(21) Appl. No.: 12/473,810

(22) Filed: May 28, 2009

Related U.S. Application Data
(60) Provisional application No. 61/130,227, filed on May 29, 2008.

(51) Int. Cl. A61K 31/472; C07D 217/02; C07D 401/12; C07C 237/40; C07D 413/12; C07D 413/14; C07D 409/12; C07D 203/16; C07D 295/12; C07D 235/04; C07D 231/56; C07D 277/62; C07D 473/00; C07D 471/04; A61K 31/4725; A61K 31/5377; A61K 31/52; A61K 31/496; A61K 31/47; A61K 31/517; A61P 25/00; A61P 25/28; A61P 25/16; A61P 9/10; A61P 17/06; A61P 27/02

(52) U.S. Cl. 514/235.2; 546/146; 564/168; 544/128; 544/363; 548/966; 544/165; 548/307.4; 548/361.1; 548/180; 544/277; 546/113; 514/307; 514/303; 514/263.1; 514/253.05; 514/312; 514/266.4

(57) ABSTRACT

The present invention is directed to compounds of generic formula (I)

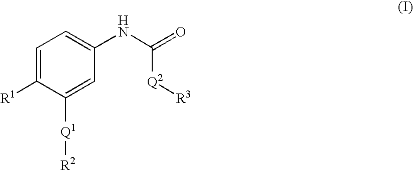

which are inhibitors of ephrin A4. The invention is also directed to pharmaceutical compositions comprising the compounds, and to the use of the compounds and compositions in the treatment of diseases regulated by the EphA4 RTK signaling, such as neurological and neurodegenerative disorders and cancer.

This application claims priority under 35 U.S.C. §119(e) of U.S. provisional application Ser. No. 61/130,227, filed May 29, 2008.

### FIELD OF THE INVENTION

The invention is directed to a class of novel compounds, their salts, pharmaceutical compositions comprising them and their use in therapy of the human body. In particular, the invention is directed to a class of EphA4 receptor tyrosine kinase inhibitors which are useful in the treatment of neurological and neurodenerative disorders, and cancer and other conditions regulated by EphA4 receptor tyrosine kinase signaling.

### BACKGROUND OF THE INVENTION

The Eph super family of receptors is the largest sub-family of receptor tyrosine kinases (RTKs), and shares 65-90% sequence homology in the kinase domain and 30-70% in the extracellular domain. At least 15 members of the Eph genes have been identified, from vertebrates, Drosophila and C. elegans. The Eph family is divided into two sub-groups, based on ligand-binding affinity and structure of the extracellular domain. The EphA receptors (A1-A9) generally bind ephrin-A members that are linked to the plasma membrane through a glycosylphosphatidylinositol anchor. The EphB (B1-B6) receptors generally bind ephrin-B members that transverse the cell membrane. Eph receptors consist of an extracellular globular domain responsible for ligand binding, a cysteine-rich region, two fibronectin type III repeats, a region spanning the cell membrane, and a tyrosine kinase domain.

Receptor-ligand binding for the Eph family is highly promiscuous within each subclass. EphA4 binds ephrinB ligands with high affinity (Murai et al, J Cell Sci 2003; 116:2823-2832). In addition, the Eph family of RTKs and their ligands, the ephrins, are membrane-bound proteins that mediate bi-directional signals between adjacent cells. Interractions between Eph receptors and ephrin ligands on adjacent cells promote the clustering of these molecules. Clustering leads to the initiation of the signal, which involves tyrosine phosphorylation mediated by Eph RTKs, and results in the activation of various intracellular signaling pathways.

Eph RTK signaling is involved in cytoskeletal organization (Murai, J Cell Sci 2003). Eph receptors also influence other signaling molecules that regulate cell behavior. In particular, Eph receptor activation has been shown to mediate cell-contact-dependent repulsion (Stein et al, Genes Dev 1998; 12:667-678). By modulating cytoskeleton dynamics affecting cell motility and adhesion, Eph receptors and ephrins orchestrate cell movements during multiple morphogenetic processes, including gastrulation, segmentation, angiogenesis, neuron axonal path finding, and neural crest cell migration (Kullander et al, Genes Dev 2001; 15:877-777, Yokoyama et al, Neuron 2001; 29; 85-97, Tessier-Lavigne, Cell 1995; 82:345-348). Continued expression of Ephs and ephrins also occurs in the cardiovascular and central nervous systems in adulthood, under physiological and pathological conditions.

### Functions in Central Nervous System Injury

Ephs and ephrins are expressed in many cell types and regions in normal adult CNS. In the white matter, Ephs and ephrins are mainly expressed on astrocytes in contact with blood vessels or closely associated with the pial surface. They are also expressed on neuronal populations in the grey matter (Wahl et al, Endothelium 9(3):205-216). Expression of Ephs and ephrins in the adult CNS also has implications for regeneration after injury. Indeed, it has been shown that many Eph receptors in adult CNS are upregulated after CNS injury (Olivieri et al, J Histochem Cytochem 47:855-861).

Following damage to the CNS, different cell types respond in different ways. Neurons typically attempt to regenerate their connections, and largely fail. Astrocytes and microglial cells proliferate, migrate and become hypertrophic. Oligodendrocytes generally attempt to remyelinate. Expression of Ephs and ephrins in each of these situations may affect the response of the cells to the damage. Given the role of Ephs and ephrins in axon guidance during development, a common view is that these molecules may also play a role in guidance during CNS regeneration. However, expression of Eph/ephrins on mature cell types, such as astrocytes and oligodendrocytes, may also have an influence that is not present during development, such as mediating astrocytic gliosis or axonal remyelination.

In vitro experiments have shown that ephrins are inhibitory and repulsive for the axonal growth of many different neuronal populations (38, 41, 46-48). The inhibitory effect on neurite outgrowth may be bidirectional involving the reverse (coming from the ephrin ligand) and/or forward (coming from the Eph receptor) signaling in neurons (Wahl et al, J Cell Biol 2000: 149:263-270).

The failure of axonal regeneration may be related to deficient regenerative capacity of adult CNS axons (Kullander et al, Neuron 2001; 29: 73-84) but also coincides with astrocytic reactivity (Goldberg et al, Science 2002 296: 1860-1864) and myelin destruction (Bouslama-Oueghlani, J Neurosci 2003; 23:8318-8329). Many studies of CNS experimental injury models have demonstrated that glial scarring is a multi-component process consisting of glial reactivity and alteration of the ECM. This reaction is the result of a multi-cellular response to injury involving astrocytes, microglia, macrophages, oligodendrocyte progenitors, fibroblasts and Schwann cells (Shewan et al, J Neurosci 1995; 15:2057-2062). Ephs and ephrins are expressed by many of these cells and may therefore contribute to their response to damage.

Glial cells are particularly sensitive markers of neuronal damage. CNS injury triggers gliosis, characterized by glial reactivity and proliferation, with morphological and functional changes in astrocytes and microglia. This astroglial response can have a dual role affecting both neuronal cell recovery and degeneration. There are some benefits of glial scar formation in the CNS, such as forming a boundary around the site of injury from the external environment, preventing infections and shrinking the lesion cavity. However, many clinical and experimental observations have shown that astrocytic gliosis and formation of the glial scar are also a major barrier to neuronal regeneration (Wahl et al, J Cell Biol 2000). Following CNS injuries, reactive astrocytes form a dense net of interdigitated processes. They upregulate inhibitory ECM molecules such as proteoglycans and tenascin, which inhibit neurite outgrowth in vitro (Jakeman et al, J Comp Neurol 1991; 307:311-334), can contribute to the physical barrier of the glial scar (Gallo et al, Dev Biol 1987; 12:282-285).

Eph receptor upregulation on astrocytes also appears to play a role in the initiation of gliosis by contributing directly or indirectly to the reactivity of the astrocytes. In EphA4 knockout mice, there was a dramatic decrease in astrogliosis and glial scar formation after spinal cord injury, whereas EphA4 expression was upregulated on astrocytes by 4 days in wild-type mice (Wahl et al, J Cell Biol 2000).

### Functions in Angiogenesis and Tumorigenesis

Angiogenesis, the formation of new blood vessels from pre-existing vasculature, is a multi-step process involving a diverse array of molecular signals. These include factors that stimulate endothelial cell proliferation, migration, and assembly, as well as recruitment of perivascular cells and extracellular matrix remodeling. Endothelial cell receptor tyrosine kinases have been recognized as critical mediators of angiogenesis.

Ephrin ligands and Eph receptors have been demonstrated to play a role in angiogenesis (Pandey et al, Science 1995 268:567-569, Daniel et al, Kidney Int Suppl 1996 57:S73-S81). The expression of ephrin ligands and Eph receptors has been shown on both arteries and veins (Adams et al, Genes Dev 1999 13:295-306). A role for Eph signaling in vascular growth and remodeling was noted when gene knockout studies of Eph receptors or ephrin ligands resulted in embryonic lethality from cardiovascular defects (Adams et al, Trends Cardiovasc Med 2000 10:183-188, Gerety et al, Development 2002 129:1397-1410). Additionally, reduction in EphA4 and EphA7 levels correlated with abnormal cellular organization of the mesenchyme and endothelium that form the umbilical arteries (Stadler et al, Development 2001 128: 4177-4188). Furthermore, in vitro studies with vascular smooth muscle cells showed that activation of the EphA4 receptor by the Ephrin A1 ligand promoted endothelial capillary-like assembly and cell attachment (Ogita et al, Circ Res 2003 93:23-27).

The growth of solid tumors is highly dependent on the ability to recruit blood vessels, which supply the tumor with growth factors and oxygen necessary for tumor survival, growth and malignancy. Although it is now clear that Eph receptors and ephrin ligands play a critical role in vascular development during embryogenesis, the function of these molecules in pathological angiogenesis has not been well characterized. A survey of expression patterns of Eph molecules in tumor vasculature revealed that the ephrinA 1 and EphA2 ligand receptor pair is consistently expressed in endothelial cells of tumor associated vessels in a variety of tumors (Ogawa et al. Oncogene 2000 19:6043-6052). More recently, it has been shown that in addition to these two members, EphA4 may play a critical role in promoting cancer cell proliferation in pancreatic carcinogenesis or development (Iizumi it al, Cancer Science 2006 97; 11:1211-1216). From the available data it is conceivable that the Eph/ephrin system plays a dual role in tumorigenesis by affecting both neovascularization and tumor cell proliferation.

These results, identifying specific functions for EphA4 RTK in injury-induced scar formation, suggest that inhibition of EphA4 RTK may provide beneficial therapeutic interventions. Thus the compounds of the invention, which are EphA4 RTK inhibitors, are believed to be useful in the treatment of neurological and neurodegenerative disorders, and cancer, and other diseases regulated by the EphA4 RTK signaling.

### SUMMARY OF THE INVENTION

The present invention is novel compounds of generic formula (I)

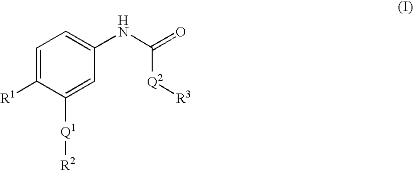

or a pharmaceutically acceptable salt thereof, which are useful as an EphA4 RTK inhibitors.

The invention is further directed to methods of treating a patient (preferably a human) for diseases or disorders regulated by the EphA4 RTK, such as neurological and neurodegenerative disorders, and cancer, by administering to the patient a therapeutically effective amount of a compound of general formula (I), or a pharmaceutically acceptable salt thereof. The invention is also directed to pharmaceutical compositions which include an effective amount of a compound of formula (I), or a pharmaceutically acceptable salt thereof, and a pharmaceutically acceptable carrier, and the use of the compounds and pharmaceutical compositions of the invention in the treatment of such diseases.

### DETAILED DESCRIPTION OF THE INVENTION

In one embodiment, the invention is directed to compounds of general formula (I)

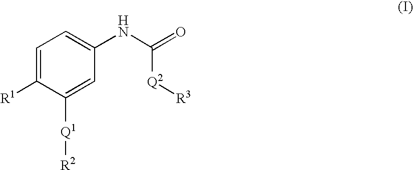

and pharmaceutically acceptable salts thereof, wherein
R¹ is selected from the group consisting of
(1) methyl, or
(2) halogen;
   Q¹ is selected from the group consisting of
(1) a bond,
(2) —NH—, or
(3) —O—C₁₋₂ alkylene-;
   R² is selected from the group consisting of
(1) —C₆₋₁₀ aryl, or
(2) heteroaryl, wherein said heteroaryl group has 5 to 12 ring atoms selected from C, N, O and S,
wherein said aryl and heteroaryl R² moiety is optionally substituted with one or more
   (a) halogen,
   (b) —C₁₋₆ alkyl,
   (c) hydroxyl,
   (d) —O—C₁₋₆ alkyl,
   (e) —SO₂—C₁₋₆ alkyl,
   (f) —C₀₋₆ alkyl-C(═O)—Oₙ—R¹⁰,
   (g) —C₂₋₆ alkenyl—C(═O)—Oₙ—R¹⁰,
   (h) —CN,
   (i) —C₀₋₆ alkyl-NR^{8A}R^{8B},
   (j) —C₀₋₆ alkyl-C(═O)—NR^{8A}R^{8B},
   (k) —NR^{9A}—C₀₋₆ alkyl-C(═O)—Oₙ—R^{9B},
   (l) heteroaryl, wherein said heteroaryl group has 5 to 12 ring atoms selected from C, N, O and S,
   wherein said alkyl, alkenyl or heteroaryl moiety is optionally substituted with one or more
      (I) halogen,
      (II) hydroxyl, or
      (III) CN;
         Q² is selected from the group consisting of
(1) a bond,
(2) —NH—(CH₂)ₙ—, or

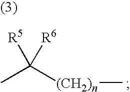

R³ is selected from the group consisting of
(1) —C₆₋₁₀ aryl,
(2) heteroaryl, wherein said heteroaryl group has 5 to 12 ring atoms selected from C, N, O and S,
(3) non-aromatic heterocyclic, wherein said heterocyclic group has 5 to 12 ring atoms selected from C, N, O and S,
(4) —C₁₋₈ alkyl,
wherein said R³ aryl, heteroaryl and non-aromatic heterocyclic moiety is optionally substituted with one or more
   (a) halogen,
   (b) —C₁₋₆ alkyl,
   (c) —C₂₋₆ alkenyl,
   (d) hydroxyl,
   (e) —O—C₁₋₆ alkyl,
   (f) —C₃₋₈ cycloalkyl,
   (g) —SO₂—C₁₋₆ alkyl,
   (h) —SO₂—NR^{8A}R^{8B},
   (i) —C₀₋₆ alkyl-C(═O)—Oₙ—R¹⁰,
   (j) —C₂₋₆ alkenyl—C(═O)—Oₙ—R¹⁰,
   (k) —CN,
   (l) —C₀₋₆ alkyl-NR^{8A}R^{8B},
   (m) —C₀₋₆ alkyl-C(═O)—NR^{8A}R^{8B},
   (n) —NR^{9A}—C₀₋₆ alkyl-C(═O)—Oₙ—R^{9B},
   (o) C₆₋₁₀ aryl,
   (p) heteroaryl, wherein said heteroaryl group has 5 to 12 ring atoms selected from C, N, O and S,
   wherein said alkyl or alkenyl moiety is optionally substituted with one or more
      (I) halogen,
      (II) hydroxyl,
      (III) CN, or
      (IV) —NR^{9A}R^{9B};
         R⁵ and R⁶ are selected from the group consisting of
(1) hydrogen,
(2) —NR^{9A}R^{9B},
(3) —C₁₋₃ alkyl,
or R⁵ and R⁶ are linked together to form a C₃₋₈ cycloalkyl group;
   R^{8A} and R^{8B} are each selected from the group consisting of
(1) hydrogen,
(2) —C₁₋₆ alkyl,
wherein said R^{8A} and R^{8B} alkyl moiety is optionally substituted with one or more halogen or NR^{9A}R^{9B},
or R^{8A} and R^{8B} are linked together to form a non-aromatic cyclic ring having from 5 to 12 ring atoms selected from C, N O and S, wherein said cyclic ring is optionally substituted with one or more C₁₋₆ alkyl;
   R^{9A} and R^{9B} are each selected from the group consisting of
(1) hydrogen, or
(2) —C₁₋₆ alkyl;
   R¹⁰ is selected from the group consisting of
(1) hydrogen, or
(2) —C₁₋₆ alkyl; and
   n is 0 or 1.

In particular embodiments, Q¹ is a bond.

In other embodiments, Q¹ is —NH—.

In other embodiments, Q¹ is —O— C₁₋₂ alkylene-, preferably —O—CH₂—.

In particular embodiments, Q² is a bond.

In other embodiments, Q² is —NH—(CH₂)ₙ—. Preferably, Q² is NH—.

In other embodiments, Q² is

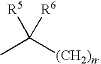

In particular embodiments of compounds of formula (I), R¹ is methyl.

In particular embodiments of compounds of formula (I), R³ is C₆₋₁₀ aryl, preferably phenyl, which is optionally substituted with one or more
(a) —C₁₋₆ alkyl,
(b) —O—C₁₋₆ alkyl,
(c) —NR^{8A}R^{8B}, or
(d) —CN,
wherein said alkyl moiety is optionally substituted with one or more halogen or CN.

In other embodiments of compounds of formula (I), R³ is heteroaryl, which is optionally substituted with one or more
(a) —C₁₋₆ alkyl,
(b)_NR^{8A}R^{8B}, or
(c) —CN,
wherein said alkyl moiety is optionally substituted with one or more halogen or CN.

In particular embodiments of compounds of formula (I), R² is C₆₋₁₀ aryl, preferably phenyl, wherein said aryl group is optionally substituted with one or more
(a) halogen,
(b) —C₁₋₆ alkyl,
(c) hydroxyl,
(d) —O—C₁₋₆ alkyl,
(e) —SO₂—C₁₋₆ alkyl,
(f) —C₀₋₆ alkyl-C(═O)—Oₙ—R¹⁰,
(g) —C₂₋₆ alkenyl—C(═O)—Oₙ—R¹⁰,
(h) —CN,
(i) —C₀₋₆ alkyl-NR^{8A}R^{8B},
(j) —C₀₋₆ alkyl-C(═O)—NR^{8A}R^{8B},
(k) —NR^{9A}—C₀₋₆ alkyl-C(═O)—Oₙ—R¹⁰,
(l) heteroaryl, wherein said heteroaryl group has 5 to 12 ring atoms selected from C, N, O and S,
wherein said alkyl or alkenyl moiety is optionally substituted with one or more
   (I) halogen,
   (II) hydroxyl, or
   (III) CN.

In other embodiments of compounds of formula (I), R² is heteroaryl, wherein said heteroaryl group has 5 to 12 ring atoms selected from C, N, O and S, wherein said heteroaryl is optionally substituted with one or more
(a) halogen,
(b) —C₁₋₆ alkyl,
(c) hydroxyl,
(d) —O—C₁₋₆ alkyl,
(e) —SO₂—C₁₋₆ alkyl,
(f) —C₀₋₆ alkyl-C(═O)—Oₙ—R¹⁰,
(g) —C₂₋₆ alkenyl—C(═O)—Oₙ—R¹⁰,
(h) —CN,
(i) —C₀₋₆ alkyl-NR^{8A}R^{8B},
(j) —C₀₋₆ alkyl-C(═O)—NR^{8A}R^{8B},
(k) NR^{9A}—C₀₋₆ alkyl-C(═O)—Oₙ—R¹⁰,
(l) heteroaryl, wherein said heteroaryl group has 5 to 12 ring atoms selected from C, N, O and S,
wherein said alkyl or alkenyl moiety is optionally substituted with one or more
   (I) halogen,
   (II) hydroxyl, or
   (III) CN.

In one embodiment, the invention is directed to methods of treating a patient (preferably a human) for diseases or disorders which are mediated by EphA4 RTK signaling, such as neurological and neurodegenerative disorders and cancer, by administering to the patient a therapeutically effective amount of a compound of general formula (I).

The invention is also directed to the use of a compound of formula (I) for treating diseases or disorders which are mediated by the EphA4 RTK, such as neurological and neurodegenerative disorders and cancer.

The invention is also directed to medicaments or pharmaceutical compositions for treating diseases or disorders which are mediated by the EphA4 RTK, such as neurological and neurodegenerative disorders and cancer, which comprise a compound of formula (I), or a pharmaceutically acceptable salt thereof, and a pharmaceutically acceptable carrier.

The invention is further directed to a method for the manufacture of a medicament or a composition for treating diseases or disorders which are mediated by the EphA4 RTK, by combining a compound of formula (I) with one or more pharmaceutically acceptable carriers.

Exemplary neurological or neurodegenerative diseases or disorders mediated by EphA4 RTK include stroke (including ischemic stroke), spinal cord injury (including paralysis induced by spinal cord injury), traumatic brain injury, and neurodegenerative diseases such as Alzheimer's Disease, Parkinson's Disease, amyotrophic lateral sclerosis, Huntington's Disease and multiple sclerosis. Other diseases or disorders for which the compounds of the invention are useful are EphA4 RTK mediated diseases or disorders such as rheumatoid arthritis, asthma, chronic obstructive pulmonary disease, Crohn's disease, psoriasis, atherosclerosis, diabetic and other retinophathies, age-related macular degeneration, neovascular glaucoma, vascular diseases, and diseases characterized by cell proliferation, including cancers. Exemplary cancer conditions include angiogenesis, tumorigenesis, and treatment of tumors, including solid malignant tumors.

The inhibitors of the EphA4 RTK may also be useful to treat diseases or disorders by promoting neuronal repair, or neuronal survival, for example by prevention or reduction of gliosis, or interference with the glial scar. The inhibitors of EphA4 RTK may also be useful to treat diseases or disorders by facilitating axonal regeneration, or to prevent inhibition of axonal growth.

Within the genus of compounds of formula (I), there is a sub-genus of compounds of formula (II):

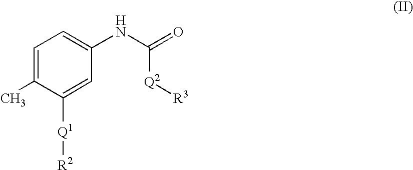

and pharmaceutically acceptable salts thereof, wherein Q¹, Q², R² and R³, are defined above.

Within the genus of compounds of formula (I), there is a sub-genus of compounds of formula (III):

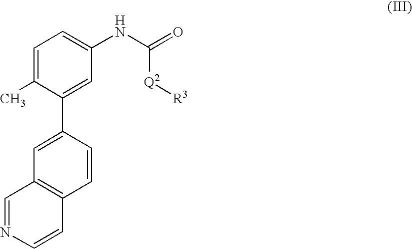

and pharmaceutically acceptable salts thereof, wherein Q² and R³ is as defined above,

Within the genus of compounds of formula (I), there is a sub-genus of compounds of formula (IV):

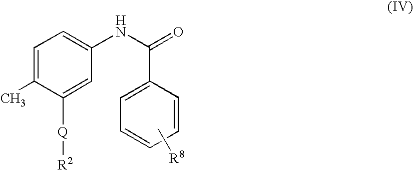

and pharmaceutically acceptable salts thereof, wherein Q¹ and R² are defined above, and R⁸ is optionally present at one or more of the phenyl carbon atoms and is selected from the group consisting of
(a) —C₁₋₆ alkyl,
(b) —O—C₁₋₆ alkyl,
(c) —NR^{8A}R^{8B}, or
(d) —CN,
wherein said alkyl moiety is optionally substituted with one or more halogen or CN.

Specific embodiments of formula (I) are described herein as Examples 1-149, or a pharmaceutically acceptable salt thereof.

The invention is also directed to methods of treating a patient (preferably a human) for diseases or disorders which are mediated by EphA4 RTK, such as neurological and neurodegenerative disorders and cancer, by administering to the patient a therapeutically effective amount of a compound of formulae (II) to (IV), or a pharmaceutically acceptable salt thereof.

The invention is also directed to the use of a compound of formulae (II) to (IV) for treating diseases or disorders which are mediated by the EphA4 RTK, such as neurological and neurodegenerative disorders and cancer, by administering to the patient a compound of formulae (II) to (IV), or a pharmaceutically acceptable salt thereof.

The invention is also directed to medicaments or pharmaceutical compositions for the treatment of diseases or disorders in a patient (preferably a human) which are mediated by the EphA4 RTK, such as neurological and neurodegenerative disorders and cancer, which comprise a compound of formulae (II) to (IV), or a pharmaceutically acceptable salt thereof, and a pharmaceutically acceptable carrier.

The invention is also directed to a method for the manufacture of a medicament or a pharmaceutical composition for treating diseases or disorders which are mediated by the EphA4 RTK, such as neurological and neurodegenerative disorders and cancer, by combining a compound of formulae (II) to (IV), or a pharmaceutically acceptable salt thereof, with a pharmaceutically acceptable carrier.

Where a variable occurs more than once in any of Formulas (I) to (IV) or in a substituent thereof, the individual occurrences of that variable are independent of each other, unless otherwise specified.

As used herein, in particular in the definitions of R², R³, R⁵, R⁶, R^{8A}, R^{8B}, R^{9A}, R^{9B} and R¹⁰, the term “alkyl,” by itself or as part of another substituent, means a saturated straight or branched chain hydrocarbon radical having the number of carbon atoms designated (e.g., C₁₋₁₀ alkyl means an alkyl group having from one to ten carbon atoms). Preferred alkyl groups for use in the invention are C₁₋₆ alkyl groups, having from one to six atoms. Exemplary alkyl groups include methyl, ethyl, n-propyl, isopropyl, n-butyl, isobutyl, tert-butyl, pentyl, hexyl, and the like. C₀ alkyl means a bond.

As used herein, in particular in the definition of R⁵ and R⁶, the term “cycloalkyl,” by itself or as part of another substituent, means a means a saturated cyclic hydrocarbon radical having the number of carbon atoms designated (e.g., C₃₋₈ cycloalkyl means a cycloalkyl group having from three to eight carbon atoms). The term cycloalkyl as used herein includes mono-, bi- and tricyclic saturated carbocycles, as well as bridged and fused ring carbocycles, such as spiro used ring systems.

Preferred cycloalkyl groups for use in the invention are monocyclic C₃₋₈ cycloalkyl groups, having from three to eight carbon atoms. Exemplary monocyclic cycloalkyl groups include cyclopropyl, cyclobutyl, cyclopentyl, cyclohexyl and the like.

As used herein, in particular in the definitions of R² and R³, the term “aryl,” by itself or as part of another substituent, means an aromatic cyclic hydrocarbon radical. Preferred aryl groups have from six to ten carbons atoms. The term “aryl” includes multiple ring systems as well as single ring systems. Preferred aryl groups for use in the invention include phenyl and naphthyl.

The term “aryl” also includes fused cyclic hydrocarbon rings which are partially aromatic (i.e., one of the fused rings is aromatic and the other is non-aromatic). An exemplary aryl group which is partially aromatic is indanyl.

As used herein, the term “halo” or “halogen” includes fluoro, chloro, bromo and iodo.

As used herein, in particular in the definition of R² and R³, the term “heteroaryl,” by itself or as part of another substituent, means a cyclic or polycyclic group having ring carbon atoms and at least one ring heteroatom (O, N or S), wherein at least one of the constituent rings is aromatic. Exemplary heteroaryl groups for use in the invention include carbazolyl, carbolinlyl, chromenyl, cinnolinyl, furanyl, benzofuranyl, benzofurazanyl, isobenzofuranyl, imidazolyl, benzimidazolyl, benzimidazolonyl, indazolyl, indolyl, isoindolyl, indolinyl, indolazinyl, indynyl, oxadiazolyl, oxazolyl, benzoxazolyl, isoxazolyl, pyranyl, pyrazinyl, pyrazolyl, benzopyrazolyl, pyridazinyl, pyridyl, pyrimidinyl, pyrrolyl, quinolyl, isoquinolyl, tetrazolyl, thiazolyl, isothiazolyl, thiadiazolyl, thienyl, benzothioenyl, benzothiazolyl, quinoxalinyl, triazinyl and triazolyl, and N-oxides thereof.

Preferred R² and R³ heteroaryl groups have from 5 to 12 ring atoms. In one such embodiment, the heteroaryl groups have 5 or 6 ring atoms.

For example, one subgroup of R² and R³ heteroaryl groups have 5 or 6 ring atoms and a single heteroatom, which is nitrogen. Exemplary heteroaryl groups in this embodiment are pyridyl and pyrrolyl.

Another subgroup of R² and R³ heteroaryl groups have 5 or 6 ring atoms and two heteroatoms, which are selected from sulfur and nitrogen. Exemplary heteroaryl groups in this embodiment are pyrazolyl, imidazolyl, thienyl and isothiazolyl.

Another subgroup of R² and R³ heteroaryl groups has 7 or 8 ring atoms and two heteroatoms, which are selected from oxygen, sulfur and nitrogen. Exemplary heteroaryl groups in this embodiment are benzoxazolyl, benzothiazolyl and quinoxalinyl.

The term “heteroaryl” also includes fused cyclic heterocyclic rings which are partially aromatic (i.e., one of the fused rings is aromatic and the other is non-aromatic). An exemplary heteroaryl group which is partially aromatic is benzodioxol.

When a heteroaryl group as defined herein is substituted, the substituent may be bonded to a ring carbon atom of the heteroaryl group, or on a ring heteroatom (i.e., a nitrogen, oxygen or sulfur), which has a valence which permits substitution. Preferably, the substituent is bonded to a ring carbon atom. Similarly, when a heteroaryl group is defined as a substituent herein, the point of attachment may be at a ring carbon atom of the heteroaryl group, or on a ring heteroatom (i.e., a nitrogen, oxygen or sulfur), which has a valence which permits attachment. Preferably, the attachment is at a ring carbon atom.

As used herein, in particular in the definition of R³, R^{8A} and R^{8B}, the term “heterocyclic” or “non-aromatic heterocyclic,” by itself or as part of another substituent, means a cycloalkyl group as defined above, in which one or more of the ring carbon atoms is replaced with a heteroatom (such as N or O). Suitable non-aromatic heterocyclic groups for use in the invention include piperidinyl, piperazinyl, morpholinyl, tetrahydropyranyl, tetrahydrofuranyl, pyrrolidinyl, pyrazolidinyl and imidazolidinyl. In certain embodiments, heterocyclic groups for use in the invention have four to eight ring atoms and a single nitrogen or oxygen heteroatom.

When a heterocyclic group as defined herein is substituted, the substituent may be bonded to a ring carbon atom of the heterocyclic group, or to a ring heteroatom (i.e., a nitrogen, oxygen or sulfur), which has a valence which permits substitution. Similarly, when a heterocyclic group is defined as a substituent herein, the point of attachment may be at a ring carbon atom of the heterocyclic group, or on a ring heteroatom (i.e., a nitrogen, oxygen or sulfur), which has a valence which permits attachment.

The compounds of the invention may have one or more asymmetric centers. Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present invention is meant to encompass all such isomeric forms of the compounds of formulae (I) to (IV).

Formulae (I) to (IV) are shown above without a definite stereochemistry at certain positions. The present invention includes all stereoisomers of formulae (I) to (VI) and pharmaceutically acceptable salts thereof.

The independent syntheses of the enantiomerically or diastereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration.

If desired, racemic mixtures of the compounds may be separated so that the individual enantiomers or diastereomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art.

Alternatively, any enantiomer or diastereomer of a compound may be obtained by stereoselective synthesis using optically pure starting materials or reagents of known configuration by methods well known in the art.

The compounds of the invention may be prepared according to the following reaction Schemes, in which variables are as defined before or are derived, using readily available starting materials, from reagents and conventional synthetic procedures. It is also possible to use variants which are themselves known to those of ordinary skill in organic synthesis art, but are not mentioned in greater detail.

In Scheme 1, isoquinoline triflate 1.1 is coupled to boronate ester 1.3 to give biaryl intermediate 1.5. Alternatively, boronate 1.2 can be prepared from 1.1 and coupled to aryl halide 1.4 to give, after Boc removal, intermediate 1.5. Coupling to either carboxylic acids or isocyanates provides amides of type 1.6 or ureas of type 1.7.

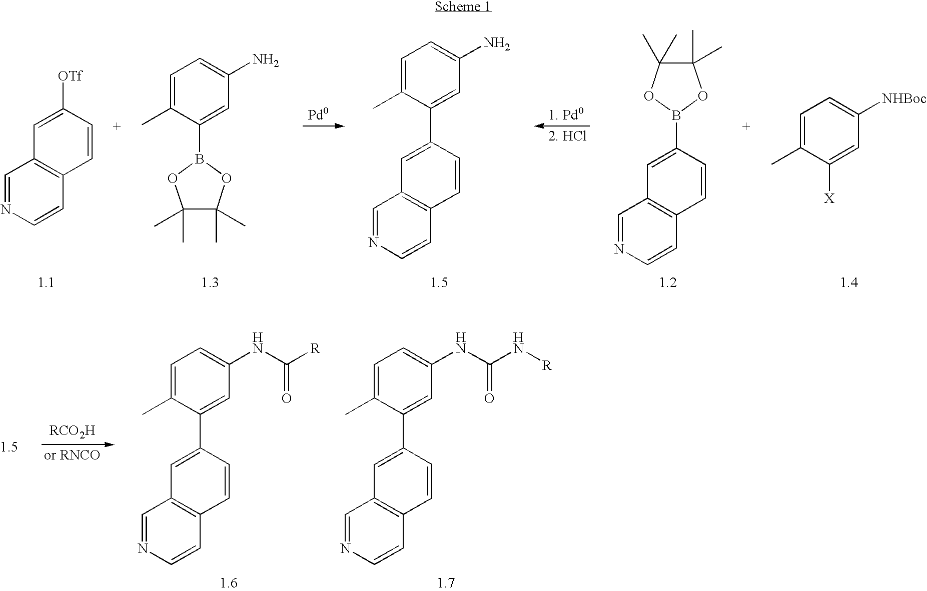

Scheme 2 describes the preparation of ortho-substituted amides of type 2.2. Functionalized benzoic acids are coupled to aniline 1.5, followed by palladium mediated functionalization of the ortho position.

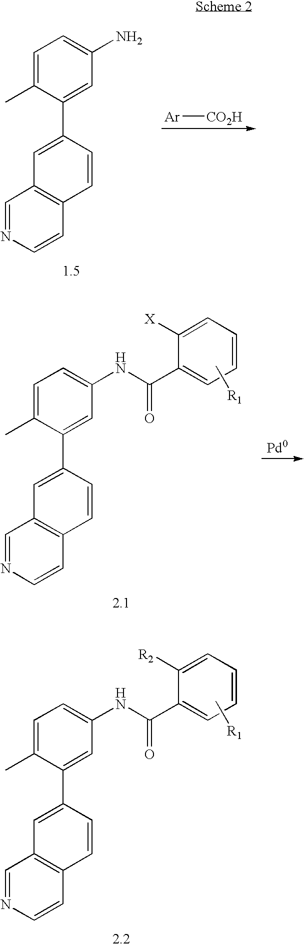

Scheme 3 describes the preparation of Eph A4 RTK inhibitors of type 3.3, 3.4, 3.6, in which the isoquinoline hinge ligand has been replaced by various groups.

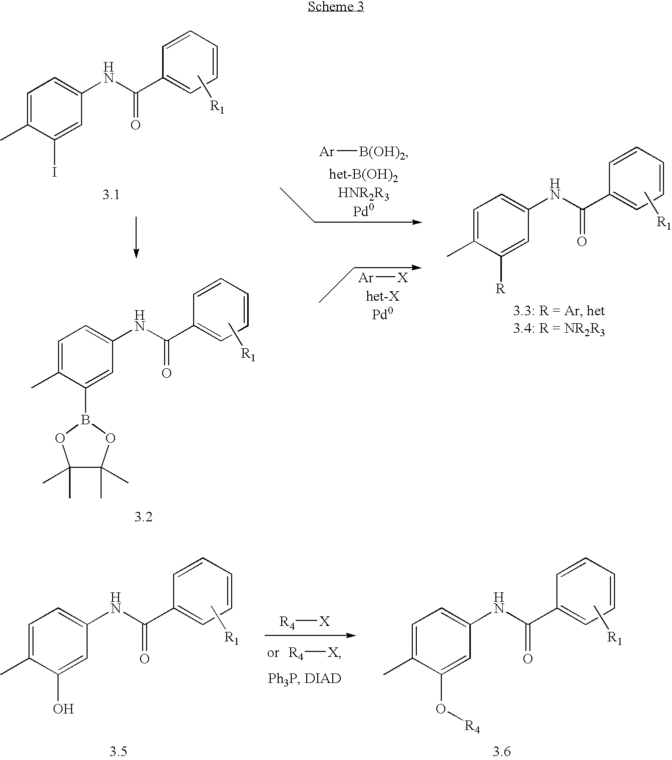

Scheme 4 describes the N-oxidation of N-containing heteroaryl (southern group). This is applicable to any compounds of types 1.6, 1.7, 2.1, 2.2, 3.3, 3.4, 3.6 containing a southern N-containing heteroaryl.

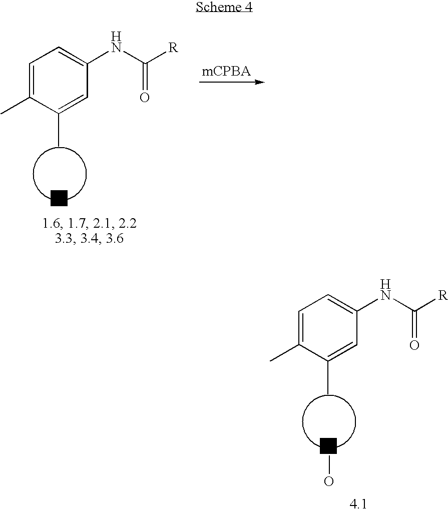

The present invention also provides a method for the synthesis of compounds useful as intermediates in the preparation of compounds of the invention.

During any of the above synthetic sequences it may be necessary or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973, and T. W. Greene & P/G. M. Wuts, Protective Groups in Organic Synthesis, John Wiley & Sons, 1991. The protecting groups may be removed at a convenient sequent stage using methods known from the art.

Specific embodiments of the compounds of the invention, and methods of making them, are described in the Examples herein.

The term “substantially pure” means that the isolated material is at least 90% pure, and preferably 95% pure, and even more preferably 99% pure as assayed by analytical techniques known in the art.

The term “pharmaceutically acceptable salts” refers to salts prepared from pharmaceutically acceptable non-toxic bases or acids including inorganic or organic bases and inorganic or organic acids. The compounds of the invention may be mono, di or tris salts, depending on the number of acid functionalities present in the free base form of the compound. Free bases and salts derived from inorganic bases include aluminum, ammonium, calcium, copper, ferric, ferrous, lithium, magnesium, manganic salts, manganous, potassium, sodium, zinc, and the like.

Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, trifluoroacetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

As used herein, the terms “EphA4 receptor tyrosine kinase” or “EphA4 RTK” are used interchangeably to refer to the high affinity cell surface receptor tyrosine kinase known as Ephrin A4. EphA4 is from the EphA sub-family of the Eph super family of receptors. The EphA receptors generally bind ephrin-A members that are linked to the plasma membrane through a glycosylphosphatidylinositol anchor. Further information about the Eph A4 receptor can be found in Murai et al, J Cell Sci 2003; 116:2823-2832.

EphA4 RTK as used herein refers to the ephrin A4 RTK of humans, or of other mammals (such as dogs, cats, mice, rats, cattle, horses, sheep, rabbits, monkeys, chimpanzees or other apes or primates).

An inhibitor of EphA4 RTK is an agent (for example, a compound of formula Ito IV herein), which demonstrates inhibition of the EphA4 RTK through one or more of the assays described herein. In some embodiments, a subject inhibitor of EphA4 RTK has an IC50 of 5 μM or less in the EphA4 kinase enzymatic assay. In some other embodiments, the compounds have an inflection point (“IP”) value of 20 μM or less (preferably 10 μM or less) in the EphA4 cell-based assay. In some other embodiments, the compounds have an IP value of 20 μM or less (preferably 10 μM or less) in the scratch wound assay. In other some embodiments, the compounds have a reduction in cell confluence of 15% or more (preferably 25% or more) in the proliferation assay.

The present invention is directed to the use of the compounds of formulas (I) to (IV) disclosed herein as EphA4 RTK inhibitors in a patient or subject such as a mammal in need of such activity, comprising the administration of an effective amount of the compound. In addition to humans, a variety of other mammals can be treated according to the method of the present invention. The subject or patient to whom the compounds of the present invention is administered is generally a human being, male or female, in whom EphA4 inhibition is desired, but may also encompass other mammals, such as dogs, cats, mice, rats, cattle, horses, sheep, rabbits, monkeys, chimpanzees or other apes or primates, for which treatment of the above noted disorders is desired.

The compounds of the present invention may be used in combination with one or more other drugs in the treatment of diseases or conditions for which the compounds of the present invention have utility, where the combination of the drugs together are safer or more effective than either drug alone. Additionally, the compounds of the present invention may be used in combination with one or more other drugs that treat, prevent, control, ameliorate, or reduce the risk of side effects or toxicity of the compounds of the present invention. Such other drugs may be administered, by a route and in an amount commonly used therefor, contemporaneously or sequentially with the compounds of the present invention. Accordingly, the pharmaceutical compositions of the present invention include those that contain one or more other active ingredients, in addition to the compounds of the present invention. The combinations may be administered as part of a unit dosage form combination product, or as a kit or treatment protocol wherein one or more additional drugs are administered in separate dosage forms as part of a treatment regimen.

In one embodiment, the compounds of the invention are useful for the treatment of stroke. Stroke occurs when normal bloodflow to the brain is disrupted, and the brain receives too much or too little blood. Ischemic stroke, which is the most common type of stroke, results from insufficient cerebral circulation of blood caused by obstruction of the inflow of arterial blood. Various disorders, including inflammation and atherosclerosis, can cause a thrombus, i.e., a blood clot that forms in a blood vessel. The thrombus may interrupt arterial blood flow, causing brain ischemia and consequent neurologic symptoms. Ischemic stroke may also be caused by the lodging of an embolus from the heart in an intracranial vessel, causing decreased perfusion pressure or increased blood viscosity with inadequate cerebral blood flow. An embolus may be caused by various disorders, including atrial fibrillation and atherosclerosis.

While not fully understood, the pathogenesis of ischemic stroke involves a complex cascade of multiple interacting biochemical events, which lead to acute neurologic injury and reduced neurological function. Brain injury from stroke leads to a cascade oif venents that can be separated ino an early phase (from the initial minutes to days), and the repair and regeneration processes of a chronic phase (from days to months).

Neuroprotective strategies targeting the early events after stroke have often failed in clinical studies. An alternative approach to stroke therapy is targeting the delayed, functional recovery. For example, damage to the central nervous system results in a glial reaction that leads to the formation of a glial scar. After cerebral ischemia, the glial scar forms an obstacle for endogenous repair mechanisms. After spinal cord injury, induction of the EPhA4 receptor plays a role in the development of the astrocyctic gliosis, which impedes axonal regeneration and inhibits functional recovery. Blockage of EphA4 RTK function may inhibit the formation of the glial scar and promote recovery after cerebral ischemia.

The compounds of the invention may be useful in combination with other agents for the treatment of stroke or stroke recovery. Examples of such second agents for treatment of stroke include, but are not limited to, aspirin, intercellular adhesion molecule (ICAM)-I and LFA-I antagonists including antibodies such as enlimomab (an anti-ICAM-1 monoclonal antibody), and anti-CD18 and anti-CD 1Ia antibodies, human anti-leukocytic antibodies such as Hu23F2G, glycoprotein lib Ilia antagonists such as eptifibatide (INTEGRELIN™), direct thrombin inhibitors, external or local ultrasound, mechanical clot retrieval or inaceration, fibrinolytic agents, neuronal wound healing agents such as basic fibroblast growth factor (e.g., FIBLAST™), neuroprotective agents such as citicoline, magnesium, nalmefene, dizocilpine, nimodipine, lamotrigine, sipatrigine, lubeluzole, mexiletine, clomethiazole, calcium and sodium channel blocking agents, beta-amino-3-hydroxy-5-methylisoxazole-4-proprionic acid antagonist, a serotonin agonist, a transmembrane potassium channel modulator, agents that inhibit astrocyte activation (e.g., ONO 2506), antioxidants (e.g., MCI-186), anti-adhesion monoclonal antibodies and antagonists and antibodies inhibiting platelet aggregation such as argatroban and abciximab (REOPRO™), phenyloin, nitrogen oxides, CNS-protective therapies, free-radical scavengers such as tirilazad, reactive oxygen metabolites, and antioxidants, and other thrombolytic agents than tenecteplase, as defined below, such as, for example, acylated plasminogen-streptokinase activator complex (APSAC), single-chain urokinase-plasminogen activator (scu-PA), thrombin-like enzymes from snake venoms such as ancrod, streptokinase (e.g., SAKSTAR™), urokinase, anistreplase, alteplase, saruplase, reteplase, lanoteplase (SUN-9216; Genetics Institute Inc.), plasmin, a truncated form of plasmin (microplasmin; ThromboGenics Ltd), a direct-acting thrombolytic with non-thrombolytic-related neuroprotective activities, recombinant desmodus rotundus salivary plasminogen activator (rDSPA) alpha-1 (Schering/Teijin Pharmaceuticals), a mutant fibrin-activated human plasminogen (BB 101 53; British Biotech Inc.), staphylokinase, fibrolase, prourokinase (intra-arterial administration directly into M1 or M2 arterial thrombus), monteplase (modified rtPA), pamiteplase, tisokinase, and vampire bat plasminogen activator, a spin-trap agent such as NXY-059 (cerovive), clopidogrel, n-methyl-dextro-aspartic acid receptor blocking agent, an anticonvulsive agent, a caspase 3 inhibitor, ((tert butylimino)methyl) 1,3 (benzenedisulfonate disodium n oxide), ebselen, glutathione peroxidase, norphenazone, rovelizumab, lactacystin beta-lactone, tsukubaenolide, 4 phosphonomethylpipecolic acid, eliprodil, antibodies to ganglioside GM1, and biologically active variants, salts, and derivatives of any of the above.

A “thrombolytic agent” is a molecule that breaks up and/or dissolves a thrombus. Exemplary thrombolytic agents include streptokinase, acylated plasminogen-streptokinase activator complex (APSAC), urokinase, single-chain urokinase-plasminogen activator (scu-PA), thrombin-like enzymes from snake venoms such as ancrod (Bell, W. “Defibrinogenating enzymes” In Colman et al (eds), Hemostasis and Thrombosis Lippincott, Philadelphia (1987) p. 886), tPA, and biologically active variants of each of the above.

The compounds of the invention are used to treat or prevent cellular proliferation diseases. Cellular proliferation disease states include, but are not limited to, cancer (further discussed below), autoimmune disease, arthritis, graft rejection, inflammatory bowel disease, proliferation induced after medical procedures, including, but not limited to, surgery, angioplasty, and the like. It is appreciated that in some cases the cells may not be in a hyper- or hypoproliferation state (abnormal state) and still require treatment. Thus, in one embodiment, the invention herein includes application to cells or individuals which are afflicted or may eventually become afflicted with any one of these disorders or states.

The compounds, compositions and methods provided herein are particularly useful for the treatment and prevention of cancer, such as angiogenesis and tumorigenesis, and including the treatment of solid tumors such as skin, breast, brain, cervical carcinomas, testicular carcinomas, and the like. Particular cancers that may be treated by the compounds, compositions and methods of the invention include, but are not limited to cardiac sarcomas: angiosarcoma, fibrosarcoma, rhabdomyosarcoma, liposarcoma, myxoma, rhabdomyoma, fibroma, lipoma and teratoma; lung sarcomas: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal sarcomas: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); genitourinary tract sarcomas: kidney (adenocarcinoma, Wilm's tumor or nephroblastoma, lymphoma, leukemia,), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver sarcomas: hepatoma (hepatocellular carcinoma), cholangiocarcinoma, hepatoblastoma, angiosarcoma, hepatocellular adenoma, hemangioma; bone sarcomas: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system sarcomas: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord (neurofibroma, meningioma, glioma, sarcoma); gynecological sarcomas: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); hematologic sarcomas: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; skin sarcomas: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. The term “cancerous cell” as provided herein, includes a cell afflicted by any one of the above-identified conditions.

In another embodiment, the compounds of the instant invention are useful for treating or preventing cancer selected from: head and neck squamous cell carcinomas, histiocytic lymphoma, lung adenocarcinoma, small cell lung cancer, non-small cell lung cancer, pancreatic cancer, papillary renal cell carcinoma, liver cancer, gastric cancer, colon cancer, multiple myeloma, glioblastomas and breast carcinoma. In another embodiment, the compounds of the instant invention are useful for the prevention or modulation of the metastases of cancer cells and cancer.

The instant compounds are also useful in combination with known anti-cancer agents. For example, the compounds are useful in combination with known anti-cancer agents. Examples of such agents can be found in Cancer Principles and Practice of Oncology by V. T. Devita and S. Hellman (editors), 6th edition (2001). Suitable anti-cancer agents include, but are not limited to, estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, and apoptosis inducing agents and agents that interfere with cell cycle checkpoints.

The instant compounds are also useful when co-administered with radiation therapy.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY1 17081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide and N-4-carboxyphenyl retinamide.

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of histone deacetylase, inhibitors of kinases involved in mitotic progression, antimetabolites, biological response modifiers, hormonal/anti-hormonal therapeutic agents, haematopoietic growth factors, monoclonal antibody targeted therapeutic agents, topoisomerase inhibitors, proteasome inhibitors and ubiquitin ligase inhibitors. Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX1OO, (trans, trans, trans)-bis-mu-(hexane-1,6-diamine)-mu-[diamine-platinum(π)]bis[diamine(chloro)platinu πi(II)]tetrachloride, diarizidinylspermine, arsenic trioxide, 1-(1 1-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755 and 4-demethoxy-3-deamino-3-aziridinyl-4-methylsulphonyl-daunorubicin.

An example of a hypoxia activatable compound is tirapazamine.

Examples of proteasome inhibitors include but are not limited to lactacystin and bortezomib.

Examples of microtubule inhibitors/microtubule-stabilising agents include paclitaxel, vindesine sulfate, 3′,4-didehydro-4′-deoxy-8′-norvincaleukoblastine, docetaxol, rhizoxin, dolastatin, mivobulin isethionate, auristatin, cemadotin, RPR 109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, epothilones (see for example U.S. Pat. Nos. 6,284,781 and 6,288,237) and BMS188797.

Examples of topoisomerase inhibitors are topotecan, hycaptamine, irinotecan, rubitecan, 6-ethoxypropionyl-3′,4′-0-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino [1,2b]quinoline-10,13(9H,15H)dione, lurtotecan, 7-[2-(N-isopropylamino)ethyl]-(20S)camptothecin, BNP1350, BNPII 100, BN80915, BN80942, etoposide phosphate, teniposide, sobuzoxane, T-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydro0xy-3,5-dimethoxyphenyl]-5 5a,6,8,8a,9-hexohydrofuro(3′,′: 6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one and dimesna.

Examples of inhibitors of mitotic kinesins include, but are not limited to inhibitors of KSP, inhibitors of MKLP1, inhibitors of CENP-E, inhibitors of MCAK, inhibitors of Kif14, inhibitors of Mphosphl and inhibitors of Rab6-KIFL.

Examples of “histone deacetylase inhibitors” include, but are not limited to SAHA, TSA, oxamflatin, PXD101, MG98, valproic acid and scriptaid. Further reference to other histone deacetylase inhibitors are described in Miller, T. A. et al. J. Med. Chem. 46(24):5097-51 16 (2003).

“Inhibitors of kinases involved in mitotic progression” include, but are not limited to, inhibitors of aurora kinase, inhibitors of Polo-like kinases (PLK) (in particular inhibitors of PLK-I), inhibitors of bub-1 and inhibitors of bub-R1.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include, but are not limited to lovastatin, simvastatin, pravastatin, fluvastatin and atorvastatin. The structural formulas of these and additional HMG-CoA reductase inhibitors that may be used in the instant methods are described at page 87 of M. Yalpani, “Cholesterol Lowering Drugs”, Chemistry & Industry, pp. 85-89 (1996). The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-H, also called Rab GGPTase).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-I (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal antiinflammatories (NSAIDs) like aspirin and ibuprofen as well as selective cyclooxy-genase-2 inhibitors like celecoxib and rofecoxib, steroidal antiinflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagi πol, thalidomide, angiostatin, troponin-1, angiotensin II antagonists and antibodies to VEGF.

Other therapeutic agents that modulate or inhibit angiogenesis and may also be used in combination with the compounds of the invention include agents that modulate or inhibit the coagulation and fibrinolysis systems (see Clin. Chem. La. Med. 38:679-692 (2000)). Examples of such agents that modulate or inhibit the coagulation and fibrinolysis pathways include, but are not limited to, heparin, low molecular weight heparins and carboxypeptidase U inhibitors (also known as inhibitors of active thrombin activatable fibrinolysis inhibitor).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen et al, Nature 2001; 4 11-0.355-365.

“Inhibitors of cell proliferation and survival signaling pathway” refer to pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of cytokine receptors, inhibitors of MET, inhibitors of PDK (for example LY294002), serine/threonine kinases (including but not limited to inhibitors of Akt, inhibitors of Raf kinase (for example BAY-43-9006), inhibitors of MEK (for example CI-1040 and PD-098059) and inhibitors of mTOR (for example Wyeth CCI-779). Such agents include small molecule inhibitor compounds and antibody antagonists.

“Apoptosis inducing agents” include activators of TNF receptor family members (including the TRAIL receptors).

The invention also encompasses combinations with NSAID's which are selective COX-2 inhibitors. For purposes of this specification, NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are 3-phenyl-4-(4-(memylsulfonyl)phenyl)-2-(5/0-furanone; a 5-chloro-3-(4-methylsulfonyl)-phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to: parecoxib, CELEBREX and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)-phenyl]methyl]-1H-1,2,3-triazole-4-carboxamide, CM 101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate) and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αv β3 integrin, to compounds which selectively antagonize, inhibit or counteract binding of a physiological ligand to the αvβ5 integrin, to compounds which antagonize, inhibit or counteract binding of a physiological ligand to both the αyβ3 integrin and the αv β3 integrin, and to compounds which antagonize, inhibit or counteract the activity of the particular integrin(s) expressed on capillary endothelial cells. The term also refers to antagonists of the αv β6, αγβ8 cti βi, 2β1<*5β1 α6β1 and α β4 integrins. The term also refers to antagonists of any combination of α β3 αv β5, cx v β6, ctv β8 ai βi, α2β1, αs β1, αββ1 and 6β4 integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin >4-(3-chloro-4-fluorophenylamino)-7-methoxy-6-[3-(4-morpholinyl)pro poxyl]quinazoline, N-(3-ethynylphenyl)-6,7-bis(2-methoxyethoxy)-4-quinazolinamine, BEBX1382, 2,3,9,10,11,12-hexahydro-10-(hydroxymethyl)-10-hydroxy-9-methyl-9,12-epoxy-1H-diindolo [1,2,3-fg:3′,2′,r-kl]pyrrolo[3,4-i][1,6]benzodiazocin-1-one, SH268, genistein, imatinib (STI571), CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine and EMD 12 1974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol 1998; 31:909-913; J. Biol. Chem. 1999; 274:91 16-9121; Invest. Opthalmol. Vis. Sd. 2000; 4 1.-2309-23 17). More recently, PPAR-γ agonists have been shown to inhibit the angiogenic response to VEGF in vitro; both troglitazone and rosiglitazone maleate inhibit the development of retinal neovascularization in mice.{Arch. Opthamol. 2001; 119:709-717).

Examples of PPAR-γ agonists and PPAR-γ/α agonists include, but are not limited to, thiazolidinediones (such as DRF2725, CS-O1 I troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NPOI10, DRF4158, NN622, G1262570, PNU1 82716, DRF552926, 2-[(5,7-dipropyl-3-trifluoromethyl-1,2-benzisoxazol-6-yl)oxy]-2-methylpropionic acid and 2(R)-7-(3-(2-chloro-4-(4-fluorophenoxy)phenoxy)propoxy)-2-ethylchromane-2-carboxylic acid.

Another embodiment of the instant invention is the use of the presently disclosed compounds in combination with gene therapy for the treatment of cancer. For an overview of genetic strategies to treating cancer see Hall et al (Am J Hum Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer.

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention maybe employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron® (dexamethasone), Kenalog®, Aristocort®, Nasalide®, Preferid®, Benecorten® or others, an antidopaminergic, such as the phenothiazines (for example prochlorperazine, fluphenazine, thioridazine and mesoridazine), metoclopramide or dronabinol. In an embodiment, an anti-emesis agent selected from a neurokinin-1 receptor antagonist, a 5HT3 receptor antagonist and a corticosteroid is administered as an adjuvant for the treatment or prevention of emesis that may result upon administration of the instant compounds.

In an embodiment, the neurokinin-1 receptor antagonist for use in conjunction with the compounds of the present invention is selected from: 2-(R)-(1-(R)-(3,5-bis(trifluoromethyl)-phenyl)ethoxy)-3-(S)-(4-fluorophenyl)-4-(3-(5-oxo-1H,4H-1,2,4-triazolo)methyl)morpholine, or a pharmaceutically acceptable salt thereof.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax®), risedronate (Actonel®), zoledronate (Zometa®), ibandronate (Boniva®), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, isoprinosine and Zadaxin®.

A compound of the instant invention may also be useful for treating or preventing breast cancer in combination with aromatase inhibitors. Examples of aromatase inhibitors include but are not limited to anastrozole, letrozole and exemestane.

A compound of the instant invention may also be useful for treating or preventing cancer in combination with siRNA therapeutics.

The compounds of the instant invention may also be administered in combination with γ-secretase inhibitors and/or inhibitors of NOTCH signaling.

A compound of the instant invention may also be useful for treating or preventing cancer in combination with PARP inhibitors.

A compound of the instant invention may also be useful for treating cancer in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); DROMOSTANOLONE PROPIONATE (DROMOSTANOLONE®); DROMOSTANOLONE PROPIONATE (MASTERONE INJECTION®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalii®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/1-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); and zoledronate (Zometa®).

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients.

In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound, which is a compound of formulae (I) to (IV), is included in an amount sufficient to produce the desired effect upon the process or condition of diseases. Accordingly, the pharmaceutical compositions of the present invention encompass any composition made by admixing a compound of the present invention and a pharmaceutically acceptable carrier.

The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, or pharmaceutically acceptable salts thereof, may also be administered by controlled release means and/or delivery devices.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Other pharmaceutical compositions include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the invention may also be in the form of oil-in-water emulsions, which may also contain excipients such as sweetening and flavoring agents.

The pharmaceutical compositions may be in the form of a sterile injectable aqueous or oleaginous suspension, or in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can also be in a form suitable for rectal administration wherein the carrier is a solid. It is preferable that the mixture forms unit dose suppositories. Suitable carriers include cocoa butter and other materials commonly used in the art.

By “pharmaceutically acceptable” it is meant the carrier, diluent or excipient must be compatible with the other ingredients of the formulation and not deleterious to the recipient thereof.

The terms “administration of” or “administering a” compound should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or P, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” means the amount of the subject compound that will elicit the biological or medical response of a tissue, system, animal or human that is being sought by the researcher, veterinarian, medical doctor or other clinician.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The compositions containing compounds of the present invention may conveniently be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples of unit dosage forms.

The compositions containing compounds of the present invention may conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

When treating or ameliorating a disorder or disease for which compounds of the present invention are indicated, generally satisfactory results are obtained when the compounds of the present invention are administered at a daily dosage of from about 0.1 mg to about 100 mg per kg of animal body weight, preferably given as a single daily dose or in divided doses two to six times a day, or in sustained release form. The total daily dosage is from about 1.0 mg to about 2000 mg, preferably from about 0.1 mg to about 20 mg per kg of body weight. In the case of a 70 kg adult human, the total daily dose will generally be from about 7 mg to about 1,400 mg. This dosage regimen may be adjusted to provide the optimal therapeutic response. The compounds may be administered on a regimen of 1 to 4 times per day, preferably once or twice per day.

The amount of active ingredient that may be combined with the carrier materials to produce a single dosage form will vary depending upon the host treated and the particular mode of administration. For example, a formulation intended for the oral administration to humans may conveniently contain from about 0.005 mg to about 2.5 g of active agent, compounded with an appropriate and convenient amount of carrier material. Unit dosage forms will generally contain between from about 0.005 mg to about 1000 mg of the active ingredient, typically 0.005, 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg, administered once, twice or three times a day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the schemes and examples herein. Starting materials are made according to procedures known in the art or as illustrated herein. The following examples are provided so that the invention might be more fully understood.

### Intermediate 1.5.1: 3-isoquinolin-7-yl-4-methylaniline (Scheme 1)

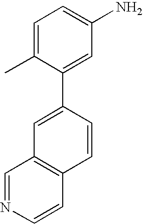

### Step A: Boronate Formation

A solution of 3-iodo-4-methyl aniline (500 mg, 2.15 mmol), bis(pinacolato)diboron (599 mg, 2.36 mmol), potassium acetate (421 mg, 4.29 mmol) in DMSO (20 mL) was degassed with nitrogen for 5 min. 1,1′-bis(diphenylphosphino)ferrocenedichloropalladium(II) DCM complex (157 mg, 0.22 mmol) was added and the solution was further degassed for 5 min. The reaction mixture was stirred, sealed, at 100° C. for 2 h, cooled to RT, diluted with aq ammonium chloride and EtOAc. The organic layer was extracted, washed with 3N LiCl, dried over sodium sulfate and concentrated in vacuo. The crude material was used as is in the next step.

### Step B: Pd Coupling

A solution of boronate from step A (2.15 mmol), isoquinolin-7-yl trifluoromethanesulfonate (654 mg, 2.36 mmol), 1M cesium carbonate (4.72 mL, 4.72 mmol) in DMF (5 mL) was degassed with nitrogen for 5 min. 1,1′-bis(diphenylphosphino)ferrocenedichloropalladium(II) DCM complex (78 mg, 0.11 mmol) was added and the solution was further degassed for 5 min. The reaction mixture was stirred, sealed, at 100° C. for 3 h, cooled to RT, diluted with aq ammonium chloride and EtOAc. The organic layer was extracted, washed with 3N LiCl, dried over sodium sulfate and concentrated in vacuo. Purification by ion exchange chromatography (SCX, MeOH to 2M NH₃/MOH) provided 3-isoquinolin-7-yl-4-methylaniline. MS M+1=235. ¹H NMR (CDCl₃, 400 MHz) δ 9.24 (s, 1H), 8.52 (d, J=6.3 Hz, 1H), 7.88 (s, 1H), 7.82 (d, J=8 Hz, 1H), 7.68 (d, J=8 Hz, 2H), 7.08 (d, J=8 Hz, 1H), 6.67 (m, 2H), 2.08 (s, 3H).

Alternatively, 3-isoquinolin-7-yl-4-methylaniline was prepared from the coupling of 7-(4,4,5,5-tetramethyl-1,3,2-dioxaborolan-2-yl)isoquinoline (obtained from isoquinolin-7-yl trifluoromethanesulfonate and bis(pinacolato)diboron using a similar procedure as described in the preparation of Intermediate 1.5.1, step A) and tert-butyl (3-bromo-4-methylphenyl)carbamate using a similar procedure as described in the preparation of Intermediate 1.5.1, step B, followed by Boc removal in HCl(g) saturated DCM.

### Intermediate 2.1.1: 2-bromo-N-(3-isoquinolin-7-yl-4-methylphenyl)-5-(trifluoromethyl)benzamide (Scheme 2)

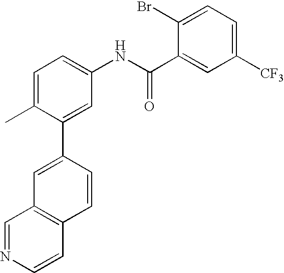

To a solution of 2-bromo-5-trifluoromethylbenzoic acid (375 mg, 1.4 mmol), 3-isoquinolin-7-yl-4-methylaniline (Intermediate 1.5.1, 327 mg, 1.4 mmol) in DMF (10 mL) was added EDC (347 mg, 1.8 mmol) and HOAt (244 mg, 1.8 mmol) and the reaction mixture was stirred at RT for 16 h, diluted with EtOAc, washed with 3N LiCl, dried over sodium sulfate and concentrated in vacuo. Purification by flash chromatography (silica 40 g, 25 to 75% EtOAc in hexane) provided 2-bromo-N-(3-isoquinolin-7-yl-4-methylphenyl)-5-(trifluoromethyl)benzamide as a pink solid. MS M+1=485. ¹H NMR (CDCl₃, 400 MHz) δ 9.27 (s, 1H), 8.53 (d, J=6.4 Hz, 1H), 7.92 (s, 2H), 7.88 (d, J=7.9 Hz, 1H), 7.82-7.76 (m, 2H), 7.76-7.66 (m, 2H), 7.62-7.54 (m, 3H), 7.34 (d, J=7.9 Hz, 1H), 2.28 (s, 3H).

Alternatively, the crude reaction mixture is purified by reverse phase preparative HPLC to provide the product as a TFA salt.

### Intermediate 3.1.1: 3-(dimethylamino)-N-(3-iodo-4-methylphenyl)benzamide (Scheme 3)

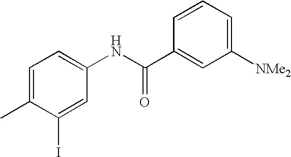

Prepared from the EDC coupling of 3-dimethylamino-benzoic acid and 3-iodo-4-methyl aniline, using a similar procedure as described for the preparation of intermediate 2.1.1. MS M+1=381.

### Intermediate 3.1.2: N-(3-iodo-4-methylphenyl)-3-(trifluoromethyl)benzamide (Scheme 3)

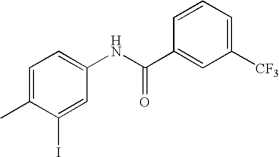

Prepared from the EDC coupling of 3-trifluoromethyl-benzoic acid and 3-iodo-4-methyl aniline, using a similar procedure as described for the preparation of intermediate 2.1.1. MS M+1=406.

### Intermediate 3.2.1: N-[4-methyl-3-(4,4,5,5-tetramethyl-1,3,2-dioxaborolan-2-yl)phenyl]-3-(trifluoromethyl)benzamide (Scheme 3)

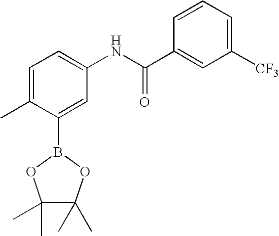

Prepared from intermediate 3.1.2 and bis(pinacolato)diboron using a similar procedure as described for the preparation of intermediate 1.5.1, step A. MS M+1=406.

### Intermediate 3.5.1: 3-(dimethylamino)-N-(3-hydroxy-4-methylphenyl)benzamide (Scheme 3)

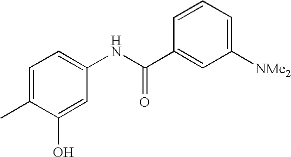

Prepared from the EDC coupling of 3-dimethylamino-benzoic acid and 3-hydroxy-4-methyl aniline, using a similar procedure as described for the preparation of intermediate 2.1.1. MS M+1=271.

TABLE 1
Examples prepared from various benzoic acids.

| | | | | ES |
|---|---|---|---|---|
| Ex # | intermediate | Mode of prep | structure | M + 1 |
| 1 | 1.5.1 | EDC, HOAt, See Intermediate 2.1.1 | 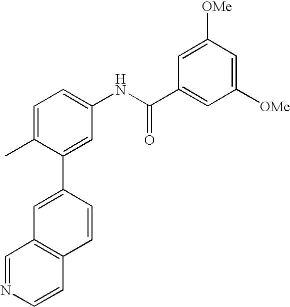 | 399 |
| 2 | 1.5.1 | EDC, HOAt, See Intermediate 2.1.1 | 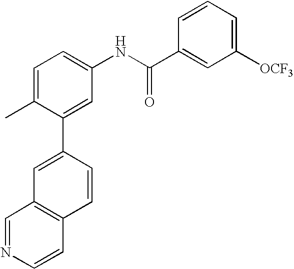 | 423 |
| 3 | 1.5.1 | EDC, HOAt, See Intermediate 2.1.1 | 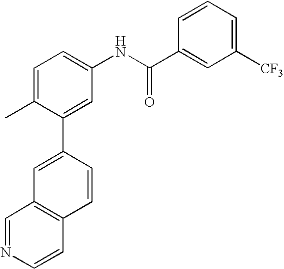 | 407 |
| 4 | 1.5.1 | EDC, HOAt, See Intermediate 2.1.1 | 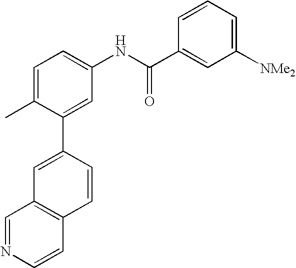 | 382 |
| 5 | 1.5.1 | EDC, HOAt, See Intermediate 2.1.1 | 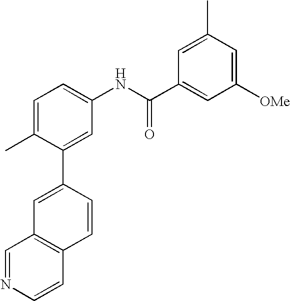 | 383 |
| 6 | 1.5.1 | EDC, HOAt, See Intermediate 2.1.1 | 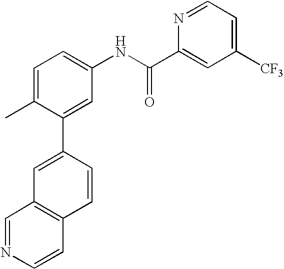 | 408 |
| 7 | 1.5.1 | EDC, HOAt, See Intermediate 2.1.1 | 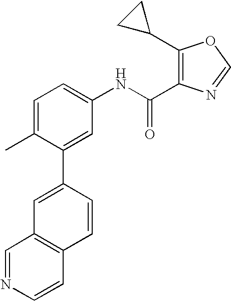 | 370 |
| 8 | 1.5.1 | EDC, HOAt, See Intermediate 2.1.1 | 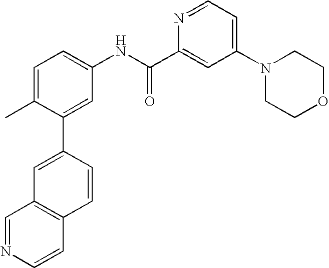 | 424 |
| 9 | 1.5.1 | EDC, HOAt, See Intermediate 2.1.1 | 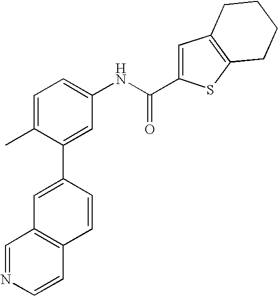 | 399 |
| 10 | 1.5.1 | EDC, HOAt, See Intermediate 2.1.1 | 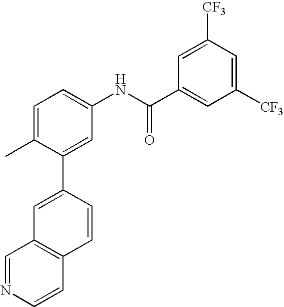 | 475 |
| 11 | 1.5.1 | EDC, HOAt, See Intermediate 2.1.1 | 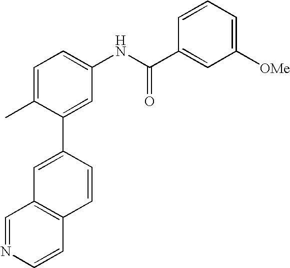 | 369 |
| 12 | 1.5.1 | EDC, HOAt, See Intermediate 2.1.1 | 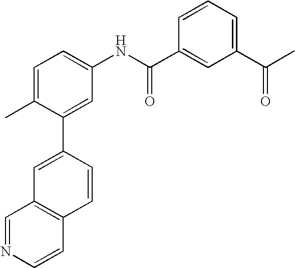 | 381 |
| 13 | 1.5.1 | EDC, HOAt, See Intermediate 2.1.1 | 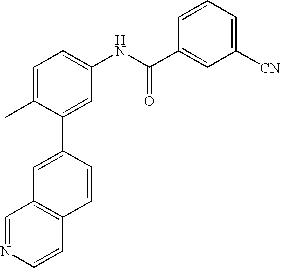 | 364 |
| 14 | 1.5.1 | EDC, HOAt, See Intermediate 2.1.1 | 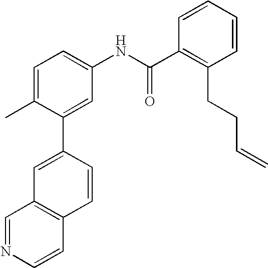 | 393 |
| 15 | 1.5.1 | EDC, HOAt, See Intermediate 2.1.1 | 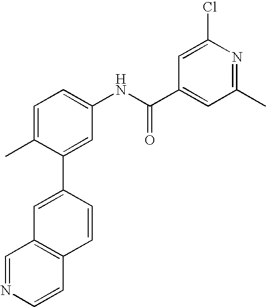 | 388 |
| 16 | 1.5.1 | EDC, HOAt, See Intermediate 2.1.1 | 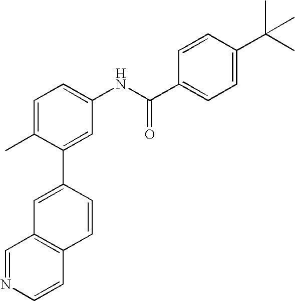 | 395 |
| 17 | 1.5.1 | EDC, HOAt, See Intermediate 2.1.1 | 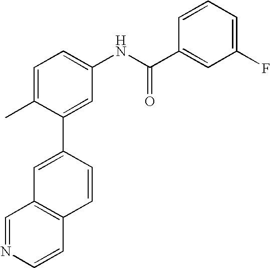 | 357 |
| 18 | 1.5.1 | EDC, HOAt, See Intermediate 2.1.1 | 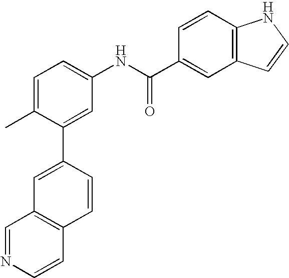 | 378 |
| 19 | 1.5.1 | EDC, HOAt, See Intermediate 2.1.1 | 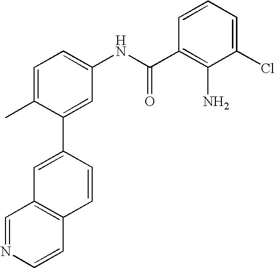 | 388 |
| 20 | 1.5.1 | EDC, HOAt, See Intermediate 2.1.1 | 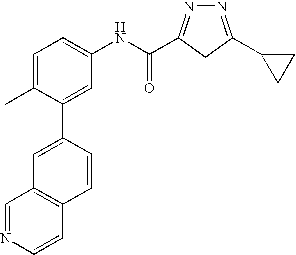 | 369 |
| 21 | 1.5.1 | EDC, HOAt, See Intermediate 2.1.1 | 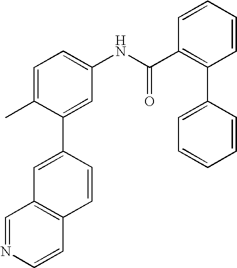 | 415 |
| 22 | 1.5.1 | EDC, HOAt, See Intermediate 2.1.1 | 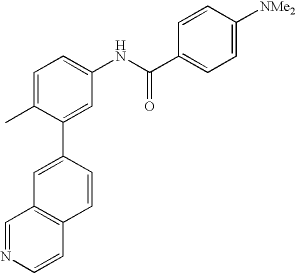 | 382 |
| 23 | 1.5.1 | EDC, HOAt, See Intermediate 2.1.1 | 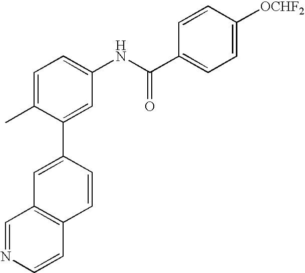 | 405 |
| 24 | 1.5.1 | EDC, HOAt, See Intermediate 2.1.1 | 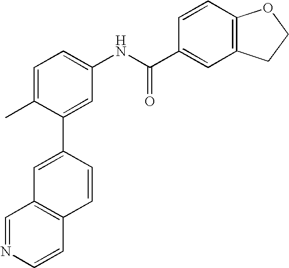 | 381 |
| 25 | 1.5.1 | EDC, HOAt, See Intermediate 2.1.1 | 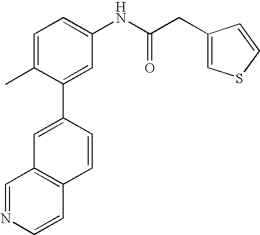 | 359 |
| 26 | 1.5.1 | EDC, HOAt, See Intermediate 2.1.1 | 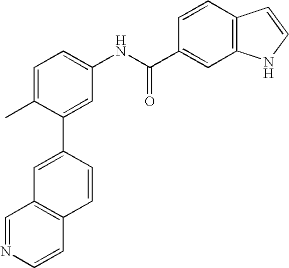 | 378 |
| 27 | 1.5.1 | EDC, HOAt, See Intermediate 2.1.1 | 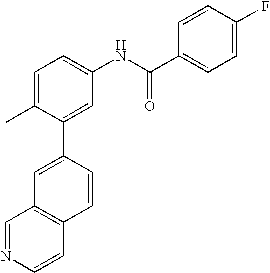 | 357 |
| 28 | 1.5.1 | EDC, HOAt, See Intermediate 2.1.1 | 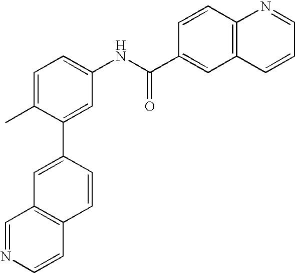 | 390 |
| 29 | 1.5.1 | EDC, HOAt, See Intermediate 2.1.1 | 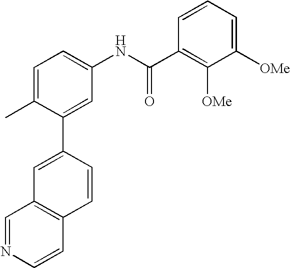 | 399 |
| 30 | 1.5.1 | EDC, HOAt, See Intermediate 2.1.1 | 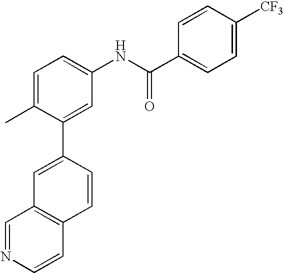 | 407 |
| 31 | 1.5.1 | EDC, HOAt, See Intermediate 2.1.1 | 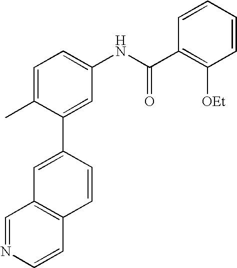 | 397 |
| 32 | 1.5.1 | EDC, HOAt, See Intermediate 2.1.1 | 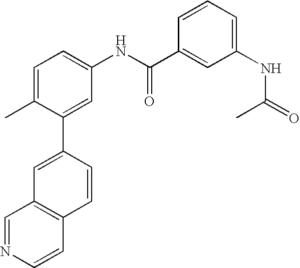 | 396 |
| 33 | 1.5.1 | EDC, HOAt, See Intermediate 2.1.1 | 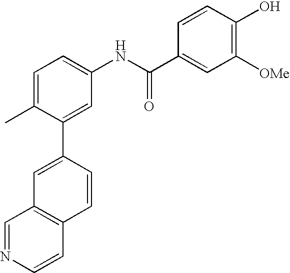 | 385 |
| 34 | 1.5.1 | EDC, HOAt, See Intermediate 2.1.1 | 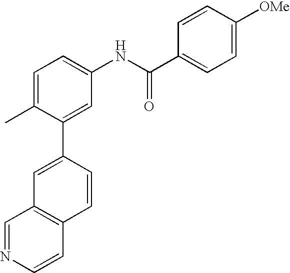 | 369 |
| 35 | 1.5.1 | EDC, HOAt, See Intermediate 2.1.1 | 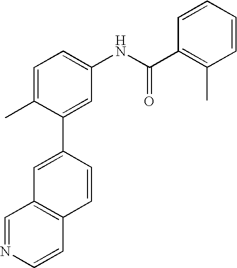 | 353 |
| 36 | 1.5.1 | EDC, HOAt, See Intermediate 2.1.1 | 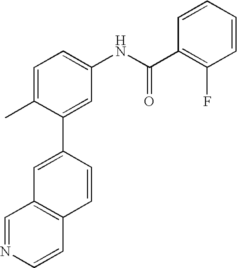 | 357 |
| 37 | 1.5.1 | EDC, HOAt, See Intermediate 2.1.1 | 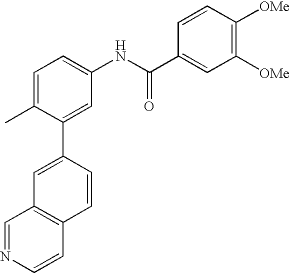 | 399 |
| 38 | 1.5.1 | EDC, HOAt, See Intermediate 2.1.1 | 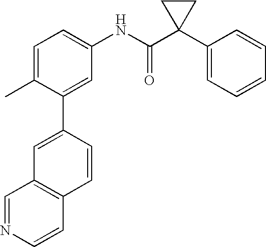 | 379 |
| 39 | 1.5.1 | EDC, HOAt, See Intermediate 2.1.1 | 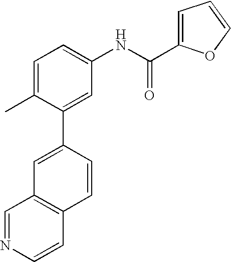 | 329 |
| 40 | 1.5.1 | EDC, HOAt, See Intermediate 2.1.1 | 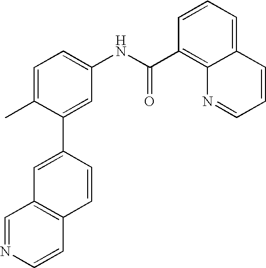 | 390 |
| 41 | 1.5.1 | EDC, HOAt, See Intermediate 2.1.1, then Boc removal | 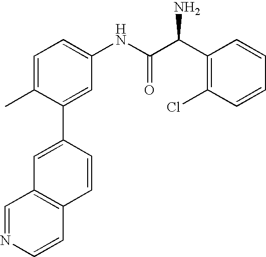 | 402 |
| 42 | 1.5.1 | EDC, HOAt, See Intermediate 2.1.1 | 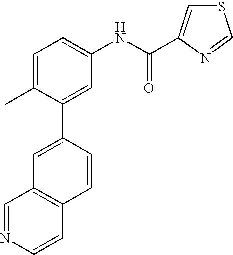 | 346 |
| 43 | 1.5.1 | EDC, HOAt, See Intermediate 2.1.1, then Boc removal | 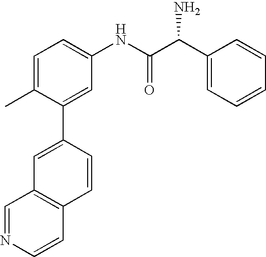 | 370 |
| 44 | 1.5.1 | EDC, HOAt, See Intermediate 2.1.1 | 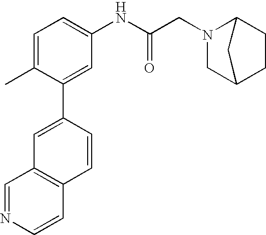 | 372 |
| 45 | 1.5.1 | EDC, HOAt, See Intermediate 2.1.1 | 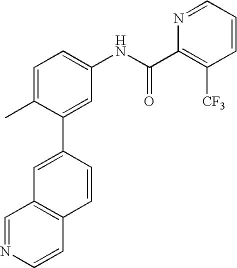 | 408 |
| 46 | 1.5.1 | EDC, HOAt, See Intermediate 2.1.1 | 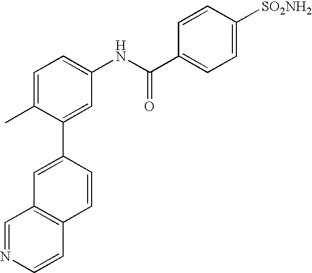 | 418 |
| 47 | 1.5.1 | EDC, HOAt, See Intermediate 2.1.1 | 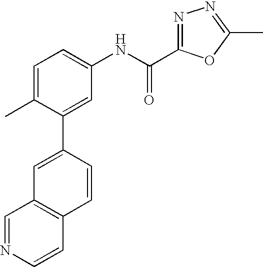 | 345 |
| 48 | 1.5.1 | EDC, HOAt, See Intermediate 2.1.1 | 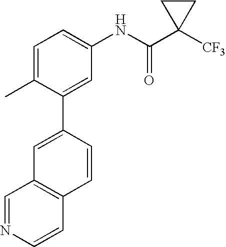 | 371 |
| 49 | 1.5.1 | EDC, HOAt, See Intermediate 2.1.1 | 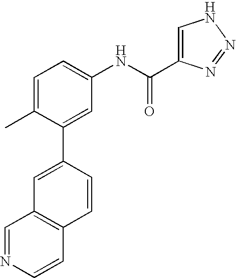 | 330 |
| 50 | 1.5.1 | EDC, HOAt, See Intermediate 2.1.1, then Boc removal | 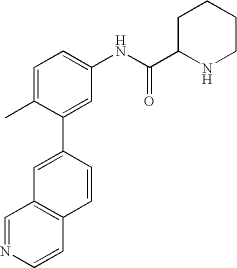 | 346 |
| 51 | 1.5.1 | EDC, HOAt, See Intermediate 2.1.1, then Boc removal | 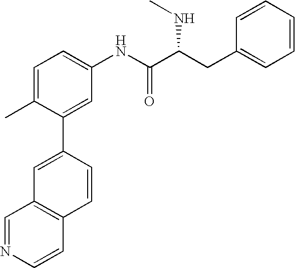 | 396 |
| 52 | 1.5.1 | EDC, HOAt, See Intermediate 2.1.1 | 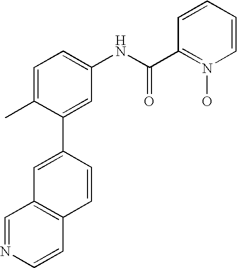 | 356 |
| 53 | 1.5.1 | EDC, HOAt, See Intermediate 2.1.1 | 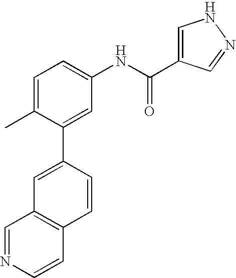 | 329 |
| 54 | 1.5.1 | EDC, HOAt, See Intermediate 2.1.1, then Boc removal | 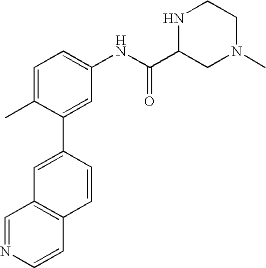 | 361 |
| 55 | 1.5.1 | EDC, HOAt, See Intermediate 2.1.1 | 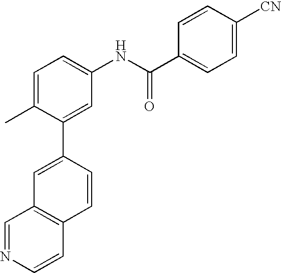 | 364 |
| 56 | 1.5.1 | EDC, HOAt, See Intermediate 2.1.1 | 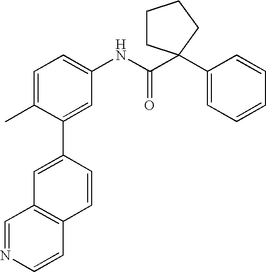 | 407 |
| 57 | 1.5.1 | EDC, HOAt, See Intermediate 2.1.1 | 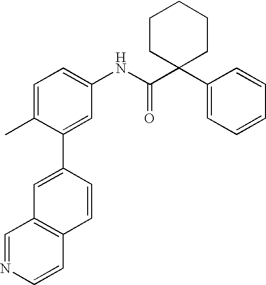 | 421 |
| 58 | 1.5.1 | EDC, HOAt, See Intermediate 2.1.1 | 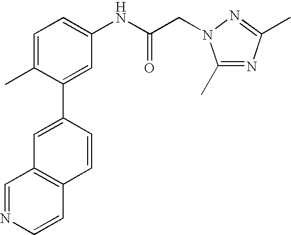 | 372 |
| 59 | 1.5.1 | EDC, HOAt, See Intermediate 2.1.1 | 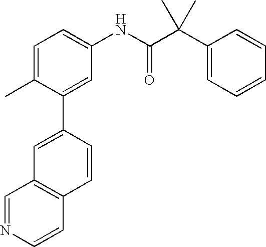 | 381 |
| 60 | 1.5.1 | EDC, HOAt, See Intermediate 2.1.1 | 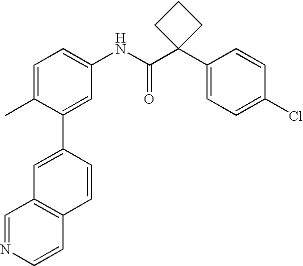 | 427 |
| 61 | 1.5.1 | EDC, HOAt, See Intermediate 2.1.1 | 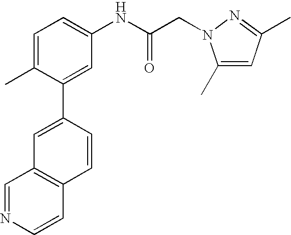 | 371 |
| 62 | 1.5.1 | EDC, HOAt, See Intermediate 2.1.1 | 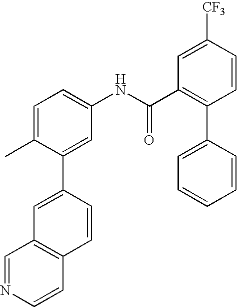 | 483 |

### Example 63

### N-(3-isoquinolin-7-yl-4-methylphenyl)-N′-phenylurea (Scheme 1)

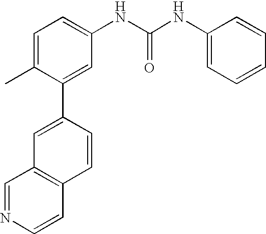

To a solution of 3-isoquinolin-7-yl-4-methylaniline hydrochloride (Intermediate 1.5.1, 15 mg, 0.06 mmol) in DCM (0.55 mL) and diisopropylethyl amine (0.01 mL, 0.06 mL) was added phenyl isocyanate (7 mg, 0.06 mmol). The reaction mixture was stirred at rt for 30 min, concentrated under a flow of nitrogen and purified by reverse phase preparative HPLC to provide N-(3-isoquinolin-7-yl-4-methylphenyl)-N-phenylurea as a TFA salt. MS M+1=354. ¹H NMR (CDCl₃, 400 MHz) δ 9.72 (s, 1H), 8.59 (d, J=7.2 Hz, 1H), 8.49-8.42 (m, 2H), 8.34 (d, J=8.1 Hz, 1H), 8.21 (d, J=8.1 Hz, 1H), 7.62 (s, 1H), 7.42 (d, J=8.1 Hz, 2H), 7.35-7.25 (m, 5H), 7.01 (t, J=8.1 Hz, 1H), 2.28 (s, 3H).

TABLE 2
Examples prepared from various isocyanates

| | | | | |
|---|---|---|---|---|
| | | | | ES |
| Ex # | intermediate | Mode of prep | Structure | M + 1 |

| | | | | |
|---|---|---|---|---|
| 64 | 1.5.1 | See ex 63 | 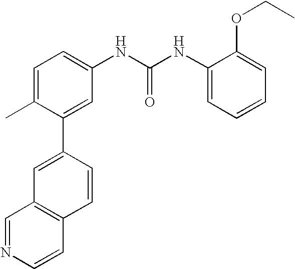 | 398 |
| 65 | 1.5.1 | See ex 63 | 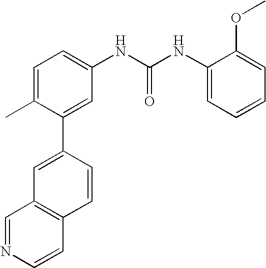 | 394 |
| 66 | 1.5.1 | See ex 63 | 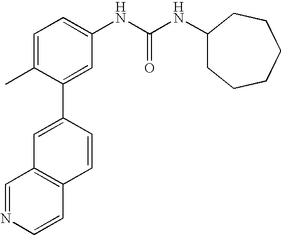 | 374 |
| 67 | 1.5.1 | See ex 63 | 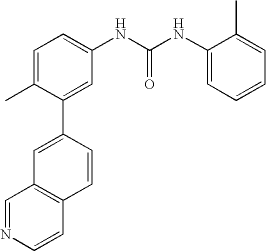 | 368 |
| 68 | 1.5.1 | See ex 63 | 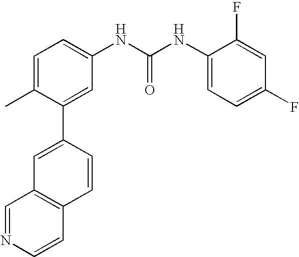 | 390 |
| 69 | 1.5.1 | See ex 63 | 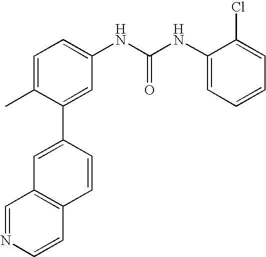 | 388 |
| 70 | 1.5.1 | See ex 63 | 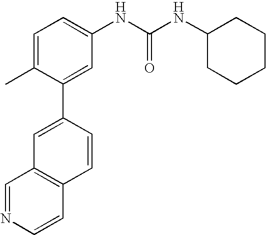 | 360 |
| 71 | 1.5.1 | See ex 63 | 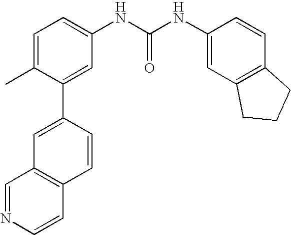 | 394 |
| 72 | 1.5.1 | See ex 63 | 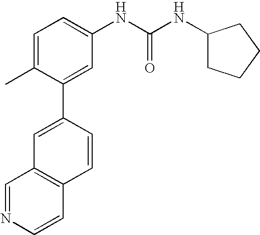 | 346 |
| 73 | 1.5.1 | See ex 63 | 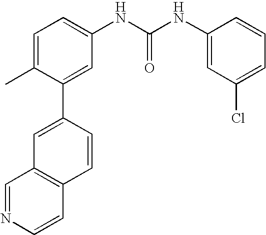 | 388 |
| 74 | 1.5.1 | See ex 63 | 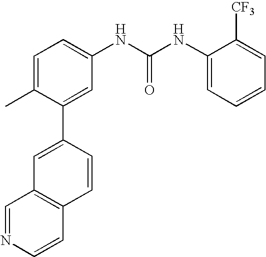 | 422 |
| 75 | 1.5.1 | See ex 63 | 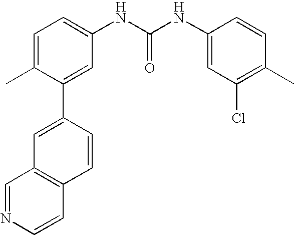 | 402 |
| 76 | 1.5.1 | See ex 63 | 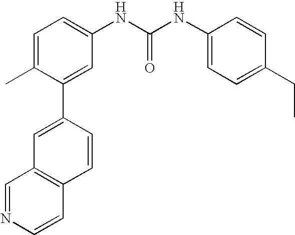 | 382 |
| 77 | 1.5.1 | See ex 63 | 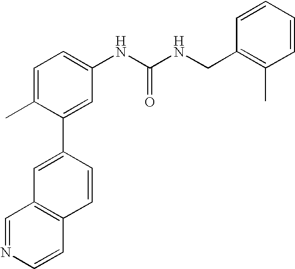 | 382 |
| 78 | 1.5.1 | See ex 63 | 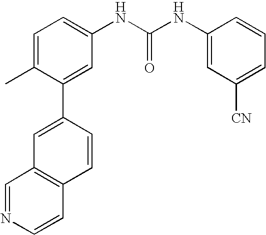 | 379 |
| 79 | 1.5.1 | See ex 63 | 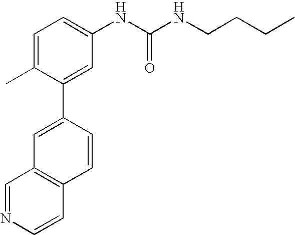 | 334 |
| 80 | 1.5.1 | See ex 63 | 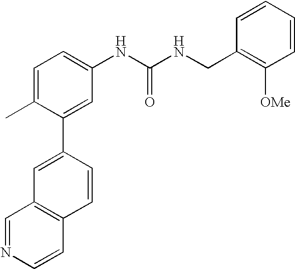 | 398 |
| 81 | 1.5.1 | See ex 63 | 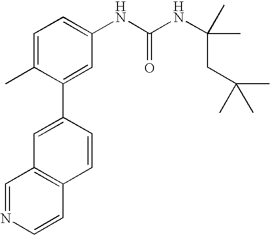 | 390 |
| 82 | 1.5.1 | See ex 63 | 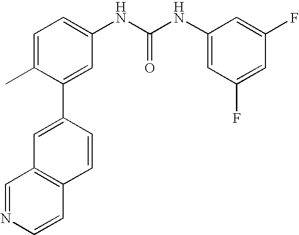 | 390 |
| 83 | 1.5.1 | See ex 63 | 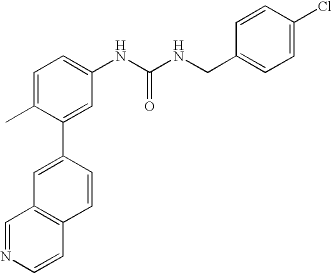 | 402 |
| 84 | 1.5.1 | See ex 63 | 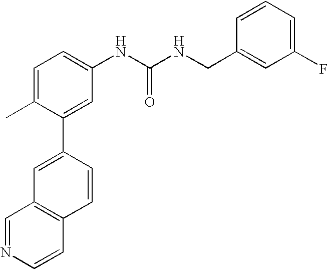 | 386 |
| 85 | 1.5.1 | See ex 63 | 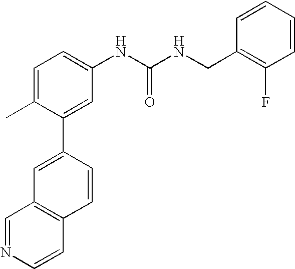 | 386 |
| 86 | 1.5.1 | See ex 63 | 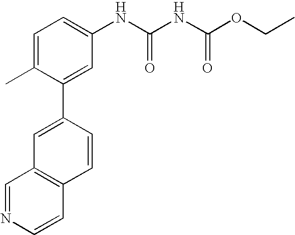 | 350 |
| 87 | 1.5.1 | See ex 63 | 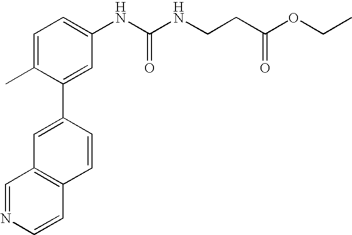 | 378 |
| 88 | 1.5.1 | See ex 63 | 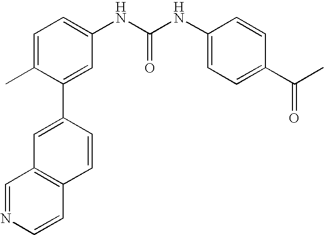 | 396 |
| 89 | 1.5.1 | See ex 63 | 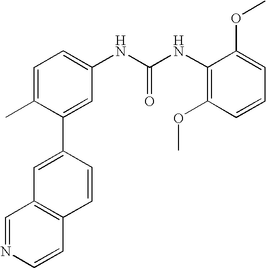 | 414 |

### Example 90

### N-(3-isoquinolin-7-yl-4-methylphenyl)-2-(4-methylpiperazin-1-yl)-5-(trifluoromethyl)benzamide (Scheme 2)

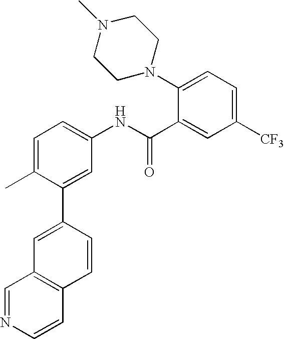

A solution of 2-bromo-N-(3-isoquinolin-7-yl-4-methylphenyl)-5-(trifluoromethyl)benzamide (intermediate 2.1.1, 30 mg, 0.06 mmol) and N-methyl piperazine (9 mg, 0.09 mmol) in THF (0.5 mL) was degassed with nitrogen for 2 min. Pd₂(dba)₃ (1 mg, 0.001 mmol), 2-dicyclohexylphosphino)-2′,4′,6′-tri-1-propyl-1,1′-biphenyl (X-PHOS, 2 mg, 0.005 mmol) and LiHMDS (31 mg, 0.19 mmol) were added in a dry environment. The reaction mixture was degassed with nitrogen for 2 min and heated to 65° C. for 16 h. The reaction mixture was concentrated in vacuo and purified by reverse phase preparative HPLC to provide N-(3-isoquinolin-7-yl-4-methylphenyl)-2-(4-methylpiperazin-1-yl)-5-(trifluoromethyl)benzamide as a TFA salt. MS M+1=505.

TABLE 2.2
Examples of type 2.2

| | | | | ES |
|---|---|---|---|---|
| Ex # | intermediate | Mode of prep | Structure | M + 1 |
| 91 | 2.1.1 | MeNH(CH₂)₂NMe₂, see example 90 | 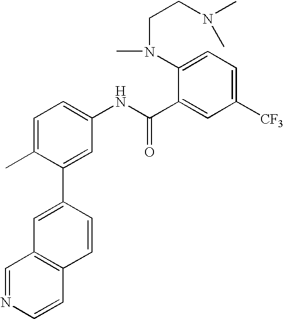 | 507 |
| 92 | 2.1.1 | MeNH(CH₂)₃NMe₂, see example 90 | 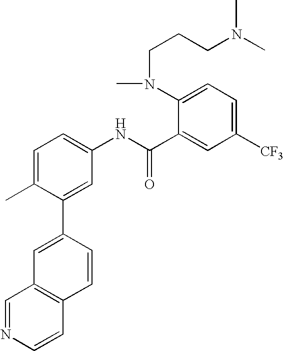 | 521 |
| 93 | 2.1.1 | Zn(CN)₂, Pd(PtBu₃)₂, Zn, DMA, 120° C. | 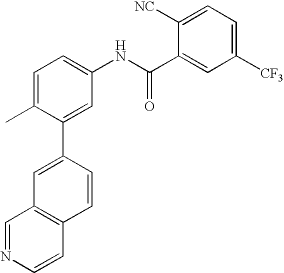 | 432 |
| 94 | 2.1.1 | NC(CH₂)₂ZnBr, see example 93 | 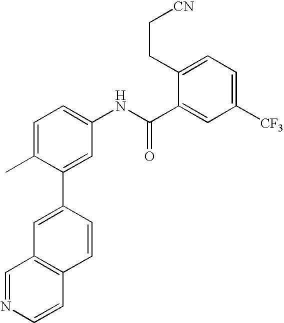 | 460 |

### Example 95

### 3-(dimethylamino)-N-(6-methyl-3′-nitrobiphenyl-3-yl)benzamide (Scheme 3)

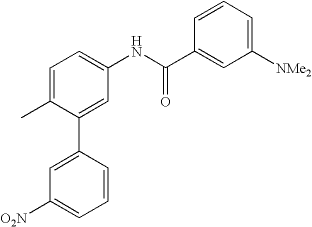

A solution of 3-(dimethylamino)-N-(3-iodo-4-methylphenyl)benzamide (intermediate 3.1.1, 45 mg, 0.12 mmol), 3-nitro-phenyl boronic acid (24 mg, 0.14 mmol) in DMF (0.5 mL) and 1M cesium carbonate (0.24 mL, 0.24 mmol) was degassed with argon for 2 min. 1,1′-bis(diphenylphosphino)ferrocene-palladium dichloride, DCM complex (4.8 mg, 0.006 mmol) was added and the reaction mixture was irradiated at 100° C. in a microwave for 10 min. The reaction mixture was filtered and purified by reverse phase preparative HPLC to provide 3-(dimethylamino)-N-(6-methyl-3′-nitrobiphenyl-3-yl)benzamide as a TFA salt. MS M+1=376.

TABLE 3
Examples prepared from various boronic acids.

| | | | | ES |
|---|---|---|---|---|
| Ex # | intermediate | Mode of prep | Structure | M + 1 |
| 96 | 3.1.1 | See example 95 | 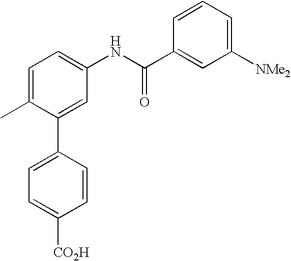 | 375 |
| 97 | 3.1.1 | See example 95 | 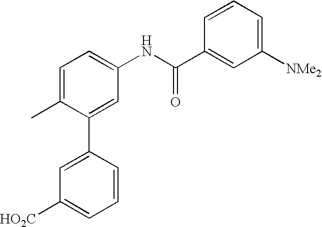 | 375 |
| 98 | 3.1.1 | See example 95 | 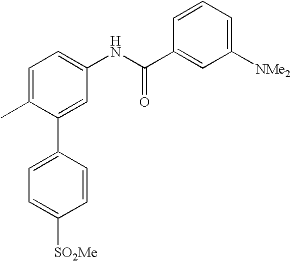 | 409 |
| 99 | 3.1.1 | See example 95 | 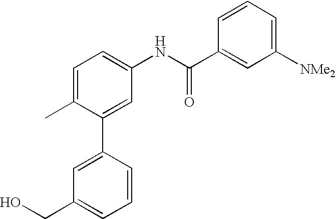 | 361 |
| 100 | 3.1.1 | See example 95 | 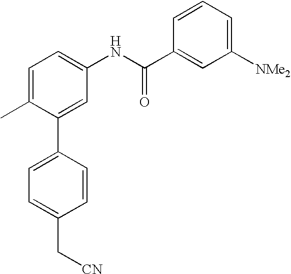 | 370 |
| 101 | 3.1.1 | See example 95 | 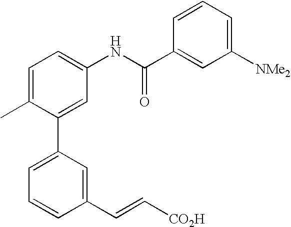 | 401 |
| 102 | 3.1.1 | See example 95 | 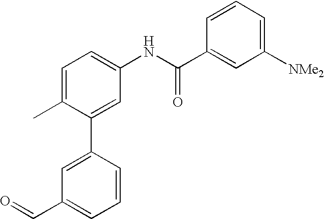 | 359 |
| 103 | 3.1.1 | See example 95 | 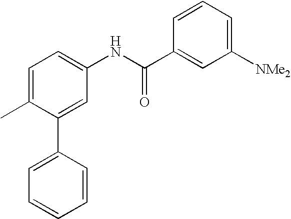 | 331 |
| 104 | 3.1.1 | See example 95 | 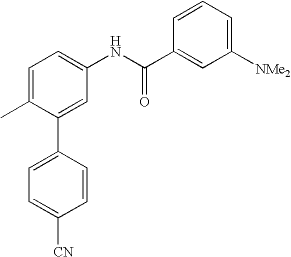 | 356 |
| 105 | 3.1.1 | See example 95 | 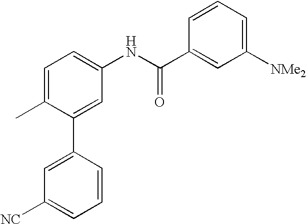 | 356 |
| 106 | 3.1.1 | See example 95 | 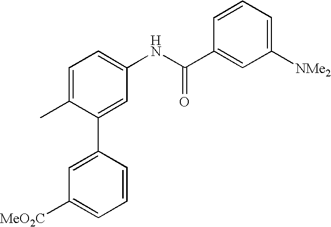 | 389 |
| 107 | 3.1.1 | See example 95 | 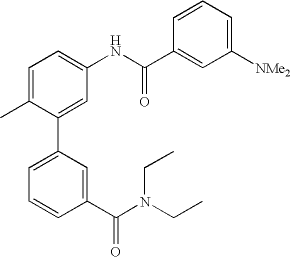 | 430 |
| 108 | 3.1.1 | See example 95 | 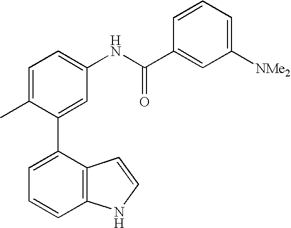 | 370 |
| 109 | 3.1.1 | See example 95 | 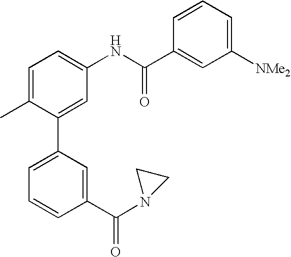 | 414 |
| 110 | 3.1.1 | See example 95 | 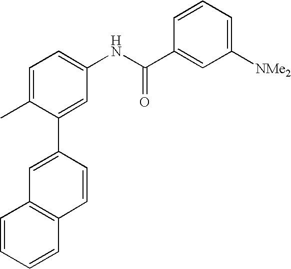 | 381 |
| 111 | 3.1.1 | See example 95 | 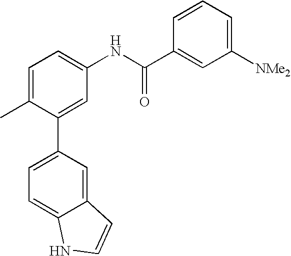 | 370 |
| 112 | 3.1.1 | See example 95 | 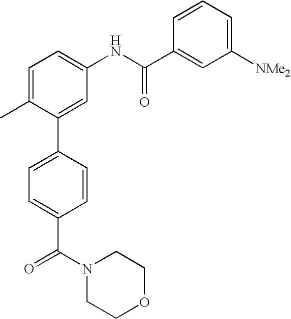 | 444 |
| 113 | 3.1.1 | See example 95 | 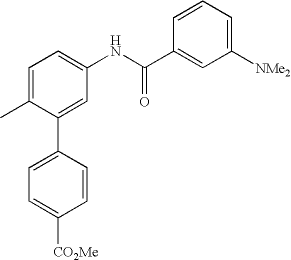 | 389 |
| 114 | 3.1.1 | See example 95 | 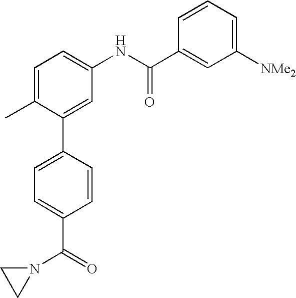 | 414 |
| 115 | 3.1.2 | See example 95 | 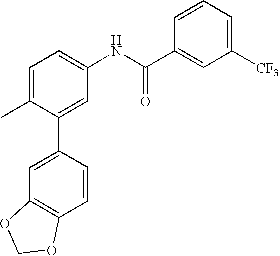 | 400 |
| 116 | 3.1.2 | See example 95 | 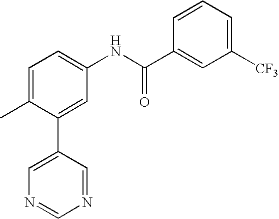 | 358 |
| 117 | 3.1.2 | See example 95 | 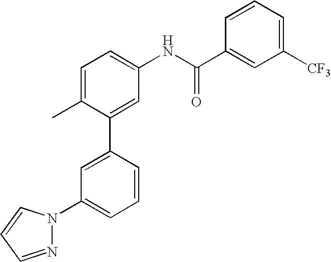 | 422 |
| 118 | 3.1.2 | See example 95 | 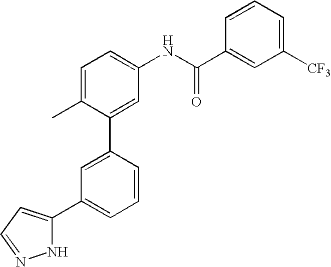 | 422 |
| 119 | 3.1.2 | See example 95 | 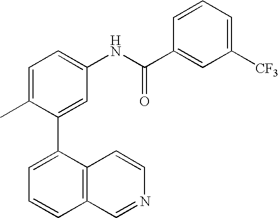 | 407 |
| 120 | 3.1.2 | See example 95 | 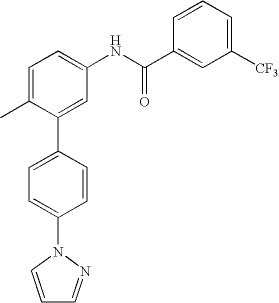 | 422 |

### Example 121

### N-[3-(3-amino-1H-indazol-6-yl)-4-methylphenyl]-3-(trifluoromethyl)benzamide (Scheme 3)

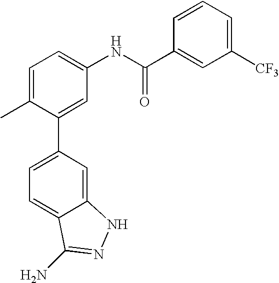

A solution of N-[4-methyl-3-(4,4,5,5-tetramethyl-1,3,2-dioxaborolan-2-yl)phenyl]-3-(trifluoromethyl)benzamide (intermediate 3.2.1, 40 mg, 0.1 mmol) and 6-bromo-1H-indazol-3-amine (23 mg, 0.11 mmol) in DMF (0.5 mL) and 1M cesium carbonate (0.25 mL) is degassed with nitrogen for 2 min. 1,1′-bis(diphenylphosphino)ferrocene-palladium dichloride, DCM complex (4 mg, 0.005 mmol) was added and the reaction mixture was irradiated at 120° C. in a microwave for 15 min. The reaction mixture was filtered and purified by reverse phase preparative HPLC to provide N-[3-(3-amino-1H-indazol-6-yl)-4-methylphenyl]-3-(trifluoromethyl)benzamide as a TFA salt. MS M+1=411.

### Example 122

### N-[3-(2-amino-1H-benzimidazol-6-yl)-4-methylphenyl]-3-(trifluoromethyl)benzamide (Scheme 3)

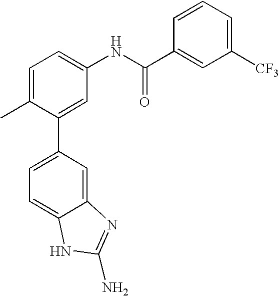

A solution of N-[4-methyl-3-(4,4,5,5-tetramethyl-1,3,2-dioxaborolan-2-yl)phenyl]-3-(trifluoromethyl)benzamide (intermediate 3.2.1, 40 mg, 0.1 mmol) and 6-chloro-1H-benzimidazol-2-amine (20 mg, 0.12 mmol) in dioxane (0.65 mL) and 1.5M KF (0.22 mL) is degassed with nitrogen for 2 min. Bis(tri-t-butylphosphine)palladium (2.5 mg, 0.005 mmol) was added and the reaction mixture was irradiated at 130° C. in a microwave for 30 min. The reaction mixture was filtered and purified by reverse phase preparative HPLC to provide N-[3-(3-amino-1H-indazol-6-yl)-4-methylphenyl]-3-(trifluoromethyl)benzamide as a TFA salt. MS M+1=411.

TABLE 4
Examples prepared from various aryl/heteroaryl bromides and chlorides

| | | | | ES |
|---|---|---|---|---|
| Ex # | intermediate | Mode of prep | structure | M + 1 |
| 123 | 3.2.1 | See example 121 or 122 | 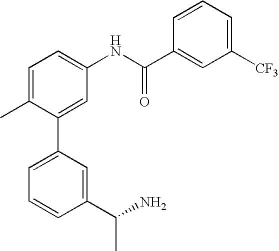 | 399 |
| 124 | 3.2.1 | See example 121 or 122 | 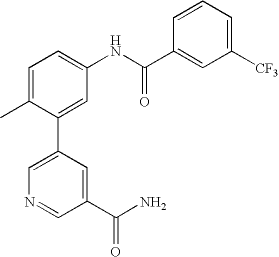 | 400 |
| 125 | 3.2.1 | See example 121 or 122 | 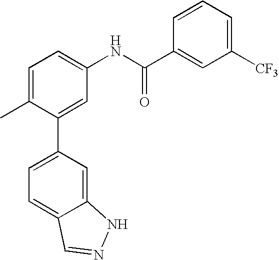 | 396 |
| 126 | 3.2.1 | See example 121 or 122 | 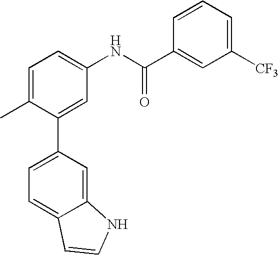 | 395 |
| 127 | 3.2.1 | See example 121 or 122 | 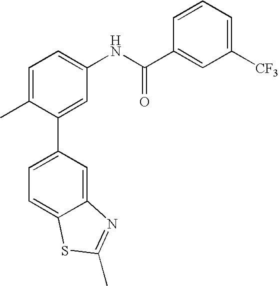 | 427 |
| 128 | 3.2.1 | See example 121 or 122 | 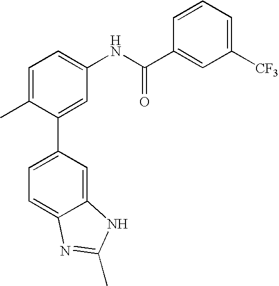 | 410 |
| 129 | 3.2.1 | See example 121 or 122 | 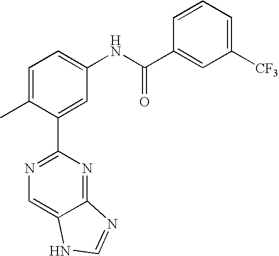 | 398 |
| 130 | 3.2.1 | See example 121 or 122 | 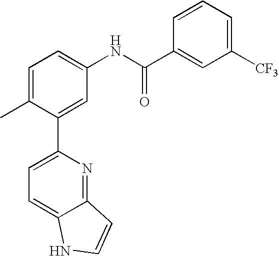 | 396 |
| 131 | 3.2.1 | See example 121 or 122 | 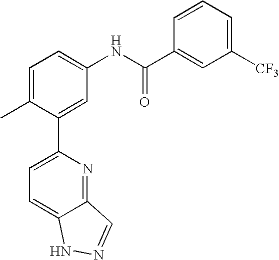 | 397 |
| 132 | 3.2.1 | See example 121 or 122 | 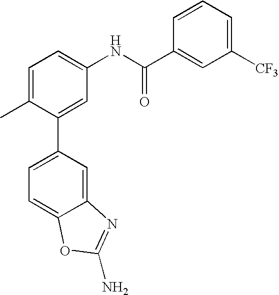 | 4112 |
| 133 | 3.2.1 | See example 121 or 122 | 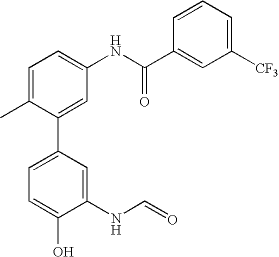 | 415 |
| 134 | 3.2.1 | See example 121 or 122 | 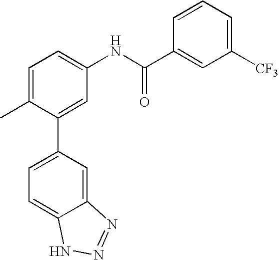 | 397 |
| 135 | 3.2.1 | See example 121 or 122 | 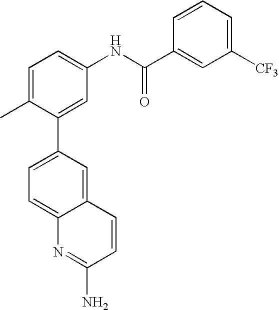 | 422 |
| 136 | 3.2.1 | See example 121 or 122 | 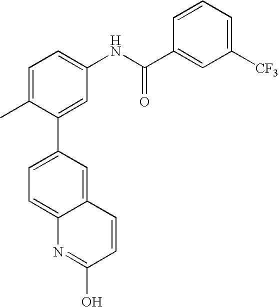 | 423 |
| 137 | 3.2.1 | See example 121 or 122 | 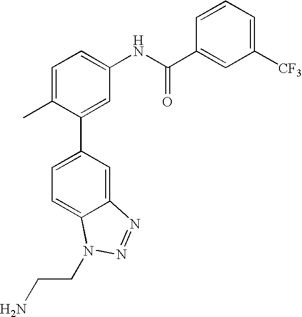 | 440 |
| 138 | 3.2.1 | See example 121 or 122 | 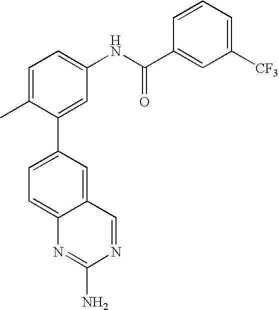 | 423 |
| 139 | 3.2.1 | See example 121 or 122 | 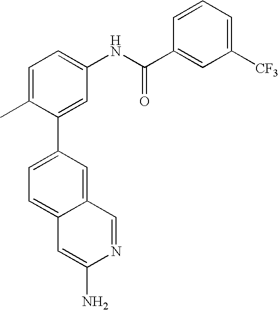 | 422 |

### Example 140

### 3-(dimethylamino)-N-{4-methyl-3-[(4-methyl-2-oxo-2H-chromen-7-yl)amino]phenyl}benzamide (Scheme 3)

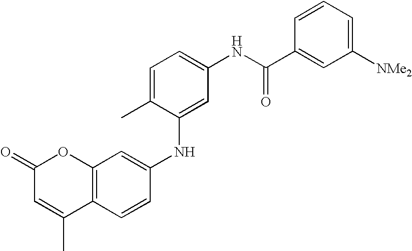

A solution of 3-(dimethylamino)-N-(3-iodo-4-methylphenyl)benzamide (intermediate 3.1.1, 40 mg, 0.11 mmol), K₃PO₄ (67 mg, 0.32 mmol) and 7-amino-4-methyl-2H-chromen-2-one (28 mg, 0.16 mmol) in DMA (0.8 mL) was degassed with nitrogen for 2 min. Bis(tri-t-butylphosphine)palladium (2.7 mg, 0.005 mmol) was added and the reaction mixture was irradiated at 120° C. in a microwave for 10 min. The reaction mixture was filtered and purified by reverse phase preparative HPLC to provide 3-(dimethylamino)-N-{4-methyl-3-[(4-methyl-2-oxo-2H-chromen-7-yl)amino]phenyl}benzamide as a TFA salt. MS M+1=428.

TABLE 5
Examples prepared from various aminoaryls and aminoheteroaryls.

| | | | | ES |
|---|---|---|---|---|
| Ex # | intermediate | Mode of prep | structure | M + 1 |
| 141 | 3.1.1 | See example 140 | 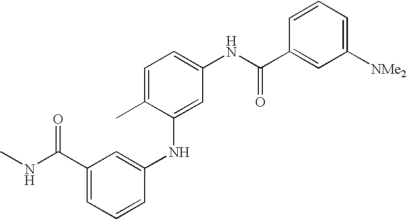 | 403 |
| 142 | 3.1.1 | See example 140 | 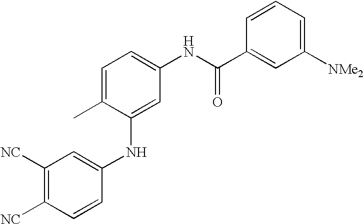 | 396 |
| 143 | 3.1.1 | See example 140 | 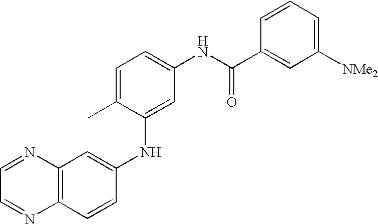 | 398 |
| 144 | 3.1.1 | See example 140 | 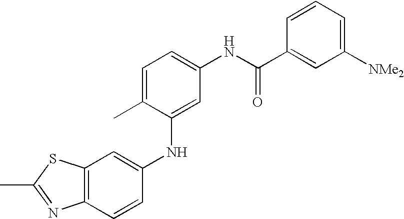 | 417 |
| 145 | 3.1.1 | See example 140 | 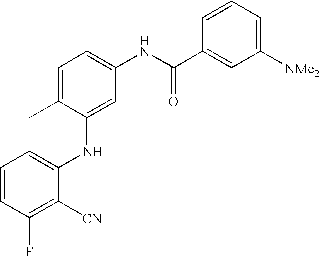 | 389 |

### Example 146.6.1

### N-[3-(benzyloxy)-4-methylphenyl]-3-(dimethylamino)benzamide (Scheme 3)

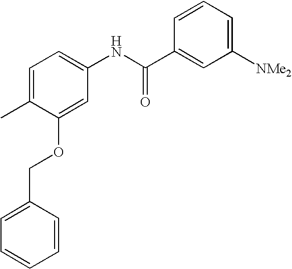

A solution of 3-(dimethylamino)-N-(3-hydroxy-4-methylphenyl)benzamide (intermediate 3.5.1, 30 mg, 0.11 mmol), benzyl bromide (0.026 mL, 0.22 mmol) and cesium carbonate (108 mg, 0.33 mmol) in DMF (0.5 mL) is stirred at rt for 16 h, filtered and purified by reverse phase preparative HPLC to provide N-[3-(benzyloxy)-4-methylphenyl]-3-(dimethylamino)benzamide as a TFA salt. MS M+1=361.

### Example 147

### 3-(dimethylamino)-N-[4-methyl-3-(2-pyridin-4-ylethoxy)phenyl]benzamide (Scheme 3)

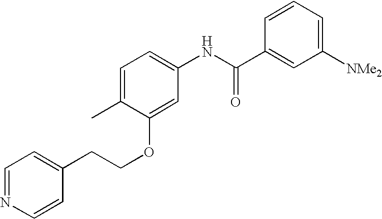

To a solution of 3-(dimethylamino)-N-(3-hydroxy-4-methylphenyl)benzamide (intermediate 3.5.1, 30 mg, 0.11 mmol), 2-pyridin-4-ylethanol (16 mg, 0.13 mmol) and triphenylphosphine (38 mg, 0.14 mmol) in THF (1 mL) was added DIAD (0.028 mL, 0.14 mmol) and the reaction mixture was stirred at rt for 16 h. The reaction mixture was concentrated in vacuo and purified by reverse phase preparative HPLC to provide 3-(dimethylamino)-N-[4-methyl-3-(2-pyridin-4-ylethoxy)phenyl]benzamide as a TFA salt. MS M+1=376.

### Example 148

### N-[3-(2,3-dihydro-1,4-benzodioxin-6-ylmethoxy)-4-methylphenyl]-3-(dimethylamino)benzamide (Scheme 3)

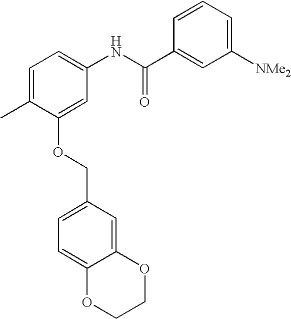

Prepared from intermediate 3.5.1 and 2,3-dihydro-1,4-benzodioxin-6-ylmethanol using a similar procedure as described in the preparation of example 85. MS M+1=419.

### Example 149

### N-(3-isoquinolin-N-oxo-7-yl-4-methylphenyl)-3-(trifluoromethyl)benzamide (Scheme 4)

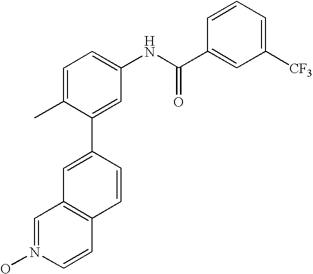

To a solution of N-(3-isoquinolin-7-yl-4-methylphenyl)-3-(trifluoromethyl)benzamide TFA salt (example 1.6.3, 10 mg, 0.02 mmol) in DCE (0.5 mL) was added mCPBA (77%, 6 mg, 0.03 mmol). The reaction mixture was stirred at rt for 1 h, at 50° C. for 2.5 h, cooled to RT. Two drops 1M Na₂S₂O₃ was added, the reaction mixture was concentrated in vacuo and purified by reverse phase preparative HPLC to provide N-(3-isoquinolin-N-oxo-7-yl-4-methylphenyl)-3-(trifluoromethyl)benzamide as a TFA salt. MS M+1=423.

### Biological Data

The utility of the compounds as EphA4 RTK inhibitors may be demonstrated by methodology known in the art, including by one or more of the assays described below.

EphA4 Kinase Enzymatic Assay

Recombinant EphA4 kinase His6-TEV-EphA4 (36 KD) (aa. 615-911) was expressed in Sf9 using baculovirus expression system. Hi-tagged protein was purified by high performance liquid chromatography in two step, using a His Trap HP column and a heparin column. The potency of compounds to inhibit EphA4 kinase phosphorylation was measured by an enzymatic assay based on time resolved fluorescence energy transfer assay format (TR-FRET), using Eu-W1024 anti-pTyr antibody (Perkin Elmer, USA) as a donor and SureLight APC (Perkin Elmer, USA) as an acceptor.

Biotinylated Poly(Glu Tyr) peptide (50 nM) was incubated in 384-well with 25 μM ATP (Sigma) and 10 ng EphA4 kinase in NEBuffer (New England Biolabs) for 60 minutes at rt. The reaction was stopped by 15 mM of EDTA solution for 10 minutes at rt. The detection mixture containing 0.5 nM Eu-W1024 anti-pTyr antibody (Perkin Elmer, USA) and 50 nM SureLight APC (Perkin Elmer, USA) was added to the wells and the plate was incubated for 30 min at rt, while protected from light. Energy transfer signal was measured using a Victor 2V (excitation filter 340, emission filter 615 and 665, delay time 50 μs). Results were expressed as ratios of the absorbencies 665/615.

Compounds were titrated in duplicate at 11 points concentration starting at 100 μM with a 3 fold increment. Compounds were diluted in DMSO. The value of 100% inhibition was measured by the ratio when the reaction was completely blocked by adding the stop reagent, EDTA, before the enzyme; whereas 0% inhibition represented the ratios in presence of DMSO only. Percentage inhibition for each compound was then calculated based on the value of the 100% inhibition. Concentration-response curves were represented and the concentrations for 50% inhibition (IC50) were calculated.

### Counterscreen Enzymatic Assays

Src Kinase and Jak2 Assays

Counterscreen assays were developed for the tyrosine kinases src kinase and jak2 kinase, with a format essentially identical to the EphA4 kinase TR-FRET format. Jak2 (50 ng) (Cell Signaling, USA) was incubated with 0.5 μM of biotinylated FLT3 peptide (Cell Signaling, USA) in presence of 5 μM of ATP (Sigma). P60c-src (10 U/well) (Upstate biotechnology, USA) was incubated with biotinylated Poly(Glu Tyr) peptide (100 nM) in presence of 0.5 μM of ATP. The enzymatic reaction was stopped after 60 min by adding 15 mM EDTA. The detection reaction and energy transfer signal measurements were similar to those previously described. Results were expressed as ratios of the absorbencies 665/615.

Compounds were titrated in duplicate at 11 points concentration starting at 100 μM with a 3 fold increment. Compounds were diluted in DMSO. The value of 100% inhibition was measured by the ratio when the reaction was completely blocked by adding the stop reagent, EDTA, before the enzyme; whereas 0% inhibition represented the ratios in presence of DMSO only. Percentage inhibition for each compound was then calculated based on the value of the 100% inhibition. Concentration-response curves were represented and the concentrations for 50% inhibition (IC50) were calculated.

P38 Alpha Kinase Assay

A counterscreen assay was performed to measure p38alpha kinase activity using the Caliper System (LifeSciences, USA). This system is based on the microfluidic technology. P38 alpha kinase activity was measured by the shift in mobility of the non-phosphorylated form when separated by electrophoresis and detected via LED (Light Emitting Diode) induced fluorescence. This assay was automated following the manufacturer's protocol. P38alpha kinase and its substrate GST-MK2 were bought from Dundee Library.

Compounds were titrated, concentration-response curves were represented and the concentrations for 50% inhibition (IC50) were calculated.

EphA4 Cell-Based Assay

A cell-based assay was used to measure the potency of the compounds on the human form of EphA4 receptor over-expressed in Chinese Hamster Ovary cell line, CHO-K1 cells (Merck Collection). The cell assay was based on a novel signaling pathway of EphA4, which involves activation of the tyrosine kinase Jak2 and the transcriptional activator Stat3 (39).

Reagents: The expression constructs of EphA4 were subcloned into the expression vector pCMV6-XL5 (Origene, Md., USA) (clone from Merck Kinase Library). The luciferase construct that was linked to the Stat1-responsive enhancer (pGAS-Luc) was bought from Stratagene (USA). The renilla construct that linked to an empty vector CMV was bought from Promega (USA).

Transfection and Luciferase Assay: CHO-K1 cells were plated in T-175 cell culture bottle in DMEM medium supplemented with 10% fetal bovine serum, 1 mM sodium pyruvate, 2 mM L-glutamine and 1.5 g/L sodium bicarbonate. When cell reached 70-80% confluence, transfections were performed in T-175 culture flasks, using Lipofectamine 2000™ transfection reagent (Invitrogen) according to the manufacturer's directions. For the luciferase assay CHO-K1 cells were co-transfected the next day with 50 mg of pGAS-Luc, 70 mg of EphA4, and 5 mg of empty vector pRL-CMV, which encoded the Renilla luciferase, and was included in the transfection mix for normalization. Six hours after transfection the cells were trypsinized and plated at 4.105 cells/ml into 96 well Black/Clear Poly-D-Lysine coated plates (Biocoat) that had been pre-spotted with compounds. Cells were lysed the next day using the Reporter Lysis Buffer (Promega), and the luciferase activity was measured using the Dual Luciferase Assay (Promega) following the manufacturer's directions. The Firefly and Renilla luminescences are read consecutively on a Top-Count (Perkin-Elmer). Results are expressed as a ratio of the two luminsescence: Firefly Luciferase/Renilla Luciferase. Cell viability was assessed on parallel plates using the Cell Glo Assay (Promega) following the manufacturer's directions. Results are expressed as percent of cell death in comparison with vehicle-treated wells.

Compounds were titrated in duplicate at 10 points concentration starting at 100 μM with a 1.5 fold increment. Concentration-response curves were represented and the concentrations at the inflexion point (IP) were calculated. The maximum effect was given by the maximum percent inhibition calculated from the results of the control wells (containing only DMSO).

Cell-Based Counterscreen Assay

EphA4 cell-based assay was based on the signaling pathway of EphA4, which involves activation of the tyrosine kinase Jak2 and the transcriptional activator Stat3. However, pharmacological agents could also interfere downstream of EphA4 signaling and particularly on Jak2 activity. To counterscreen this potential effect unrelated to EphA4 activity, a new assay was designed based on direct activation of the tyrosine kinase Jak2 and the transcriptional activator Stat3 with Interferon Gamma (IFN-γ).

MCF-7 cells were maintained in MEM growth media, supplemented with 10% fetal bovine serum, 2 mM L-glutamine, 1.5 g/L sodium bicarbonate, 0.1 mM non-essential amino acids, 1 mM sodium pyruvate, and 0.01 mg/ml bovine insulin. For the luciferase assay MCF-7 cells were co-transfected the next day with 50 mg of pGAS-Luc and 5 mg of empty vector pRL-CMV, which encoded the Renilla luciferase, and was included in the transfection mix for normalization. Six hours after transfection the cells were trypsinized and plated at 4.105 cells/ml into 96 well Black/Clear Poly-D-Lysine coated plates (Biocoat) that had been pre-spotted with compounds. IFN-γ (BD Pharmingen, USA) was then added to the wells. Cells were lysed the next day using the Reporter Lysis Buffer (Promega), and the luciferase activity was measured using the Dual Luciferase Assay (Promega) following the manufacturer's directions. The Firefly and Renilla luminescences are read consecutively on a Top-Count (Perkin-Elmer).

Results are expressed as a ratio of the two luminsescence: Firefly Luciferase/Renilla Luciferase. Cell viability was assessed on parallel plates using the Cell Glo Assay (Promega) following the manufacturer's directions. Results are expressed as percent of cell death in comparison with vehicle-treated wells.

Compounds were titrated in duplicate at 10 points concentration starting at 100 μM with a 1.5 fold increment. Concentration-response curves were represented and the concentrations at the inflexion point (IP) were calculated. The maximum effect was given by the maximum percent inhibition calculated from the results of the control wells (containing only DMSO).

Scratch Wound Assay

Cell Cultures: C2C12 mouse myoblast cell line (ATCC, VA, USA) was maintained in culture in DMEM media supplemented with 10% fetal bovine serum, 1 mM sodium pyruvate, 1.5 g/L sodium bicarbonate, and 100 IU of penicillin, 100 mg/ml of streptomycin. The cells were then differentiated into myotubes with DMEM supplemented with 2% horse serum for 3 days in 24 well ImageLock™ plate (Essen Instruments, Mich., USA).

Primary Rat cortical astrocytes were purchased from Lonza (Walkersvelle Inc., MA, USA). Cells were plated in culture flasks at a density of 1.106-3.106 cells/ml, and maintained at 37° C. and 95% CO₂/5% O₂, in Astrocyte Growth Medium Bulletkit™ (Lonza Walkersville Inc., MA, USA). When astrocyte cultures reached confluence, cells were trypsinized and replated at 6.105 cells/ml onto 24 well ImageLock™ plate (Essen Instruments, Mich., USA).

Scratch wound assays are commonly used to assess the effects of drugs and drug candidates on the cellular proliferation and/or migration associated with wound closing. The IncuCyte™ system (Essen Instruments, Mich., USA), an automated imaging platform, provides ongoing, real-time images and quantitative data generated throughout the wound-closing process. Cells were maintained in 24 well ImageLock™ (Essen Instruments, Mich., USA) at 37° C. and 95% CO₂/5% O₂, until a confluent monolayer was achieved. A single scratch wound was then created in each well using the Essen Woundmaker™ (Essen Instruments, Mich., USA). It induced a mechanical scratch of the cell monolayer using plastic pipette tips (10-20 μl pipette tips, Eppendorff). Dead cells and other debris produced by the scratch were immediately washed with regular growth media. Cells were then treated with pharmacological agents and were placed into the IncuCyte™ at 37° C. and 95% CO₂/5% O₂, for two to three days. During this time, the wound area within each well was repeatedly imaged at fixed time intervals (every three hours). The IncuCyte™ scratch wound software generates a “wound mask” for each well. An initial wound mask is created for the first image (at time 0) that clearly delineates the border of the wound. A revised mask is generated for each subsequent image to track wound closure. Wound closure can be automatically monitored using the wound confluence (% confluence). Wound confluence can be graphed over time to evaluate the characteristics of wound closing in the presence of pharmacological agents.

The calculation of the area under the curve (AUC) of the % confluence function of the time gave a quantitative measurement of compounds efficacy on the scratch wound closing. Compounds were titrated in quadruplicate at 5 points concentration starting at 20 nM with a two-fold increment. The percentage of inhibition was calculated from the AUCs at each dose in comparison with the AUCs of the control wells (1% DMSO). Concentration-response curves were represented and the concentrations at the inflexion point (IP) were calculated. The maximum effect was given by the maximum percent inhibition.

Proliferation Assay

C2C12 were plated at 15,000 cells per well and then differentiated into myotubes with DMEM supplemented with 2% horse serum for 3 days in 24 well ImageLock™ plate (Essen Instruments, Mich., USA). Primary Rat cortical astrocytes were plated at 3.105 cells/ml onto 24 well ImageLock™ plate.

Cells were placed into the IncuCyte™ at 37° C. and 95% CO₂/5% O₂, for two to three days. During this time, the cells within each well were repeatedly imaged at fixed time intervals (every three hours). The IncuCyte™ system automatically monitored the proliferation using the percentage of confluence. Percent of confluence can be graphed over time to evaluate the characteristics of proliferation in the presence of pharmacological agents.

Compounds were tested at 5 nM in quadruplicate. The percentage of reduction of cell confluence was calculated from the AUCs in comparison with the AUCs of the control wells (1% DMSO).

TABLE
Inhibition Profiles of Exemplary Inhibitors of EphA4 RTK

EXAMPLES

| ASSAY | Example 120 | Example 133 |
|---|---|---|
| EphA4 Kinase IC50 (μM) | 1.59 (70%) | 4.8 (93%) |
| (max inhibition in %) | | |
| EphA4 Cell Assay IP (μM) | 5.6 (50%) | 8.3 (42%) |
| Max Toxicity Cell Assay | 8% at 25 μM | 15% at 50 μM |
| % of cell death | | |
| Cell Counter Screen Assay IP | >100 | 1.7 (99%) |
| (μM) (max inhibition in %) | | |
| Scratch Assay C2C12 Cells IP | 3.1 (43%) | 6 (42%) |
| (μM) (max inhibition in %) | | |
| % Reduction Confluence | 33.3% at 5 μM | 17.5% at 5 μM |
| C2C12 Cells | | |
| Max Toxicity C2C12 in cells | 10% 20 μM | 10% 20 μM |
| % of cell death | | |
| Scratch Assay Astrocytes IP | n.d. | 5 (50%) |
| (μM) (max inhibition in %) | | |
| % Reduction of Confluence | n.d. | 28.2% at 5 μM |
| Astrocytes | | |
| Max Toxicity Astrocytes | n.d. | 0% 20 μM |
| % of cell death | | |
| Src Kinase IC50 (μM) | >100 | >100 |
| Jak2 Kinase | >100 | >100 |
| IC50 (μM) | | |
| P38 Kinase IC50 (μM) | n.d. | 6.9 |

EphA4 kinase inhibition is assessed by both an enzymatic assay measuring the phosphorylation of a purified recombinant EphA4 kinase and a cell-based assay using the activation of pGas-Luciferase system in EphA4-transfected CHO-K1 cells. The functional effects of EphA4 RTK inhibitors on cell motility and proliferation is evaluated by the scratch wound assay. The effect of EphA4 RTK inhibitors is also directly measured on the cell confluence. The counterscreen assays (cell counterscreen, Src, Jak2 and P38α kinase) identified other potential inhibitory activities of compounds unrelated to EphA4 kinase. The toxicity is measured by the percent of cell death in the different cell assays. Values represent IC50 and IP in μM calculated from the dose-response curves. Values in parentheses give the maximum percent inhibition.

The following abbreviations are used throughout the text:

Me: methyl

Et: ethyl

t-Bu: tert-butyl

Ac: acetyl

Ar: aryl

Ph: phenyl

Bn: benzyl

DCE: dichloroethylene

BOC: t-butyloxycarbonyl

HMDS: hexamethyldisilazane

DIAD: diisopropyl azodicarboxylate

DMA: N,N-dimethylacetamide

TFA: trifluoroacetyl

HOAt: 1-hydroxy-7-azabenzotriazole

EDC: 1-Ethyl-3-(3-dimethyllaminopropyl)carbodiimide

DCM: dichloromethane

DMF: N,N-dimethylformamide

Dba: dibutylamine

LiHMDS: lithium hexamethyldisilazide

THF: tetrahydrofuran

DMSO: dimethylsulfoxide

DMEM: Dulbecco's Modified Eagle Medium (High Glucose)

FBS: fetal bovine serum

rt: room temperature

min: minutes

aq: aqueous

HPLC: high performance liquid chromatography

MS: mass spectrometry

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

What is claimed is:

1. A compound of formula (I) 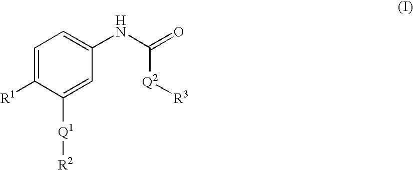
or a pharmaceutically acceptable salt thereof, wherein
R¹ is selected from the group consisting of
(1) methyl, or
(2) halogen;
Q¹ is selected from the group consisting of
(1) a bond,
(2) —NH—, or
(3) —O—C₁₋₂ alkylene-;
R² is selected from the group consisting of
(1) —C₆₋₁₀ aryl, or
(2) heteroaryl, wherein said heteroaryl group has 5 to 12 ring atoms selected from C, N, O and S,
wherein said aryl and heteroaryl R² moiety is optionally substituted with one or more
(a) halogen,
(b) C₁₋₆ alkyl,
(c) hydroxyl,
(l) —O—C₁₋₆ alkyl,
(m) —SO₂—C₁₋₆ alkyl,
(f) —C₀₋₆ alkyl-C(═O)—Oₙ—R¹⁰,
(g) —C₂₋₆ alkenyl—C(═O)—Oₙ—R¹⁰,
(h) —CN,
(i) —C₀₋₆ alkyl-NR^{8A}R^{8B},
(j) —C₀₋₆ alkyl-C(═O)—NR^{8A}R^{8B},
(k) NR^{9A}—C₀₋₆ alkyl-C(═O)—Oₙ—R^{9B},
(l) heteroaryl, wherein said heteroaryl group has 5 to 12 ring atoms selected from C, N, O and S,
wherein said alkyl, alkenyl or heteroaryl moiety is optionally substituted with one or more
(I) halogen,
(II) hydroxyl, or
(III) CN;
Q² is selected from the group consisting of
(1) a bond,
(2) —NH—(CH₂)ₙ—, or 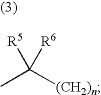
R³ is selected from the group consisting of
(1) —C₆₋₁₀ aryl,
(2) heteroaryl, wherein said heteroaryl group has 5 to 12 ring atoms selected from C, N, O and S,
(3) non-aromatic heterocyclic, wherein said heterocyclic group has 5 to 12 ring atoms selected from C, N, O and S,
(4) —C₁₋₈ alkyl,
wherein said R³ aryl, heteroaryl and non-aromatic heterocyclic moiety is optionally substituted with one or more
(a) halogen,
(b) —C₁₋₆ alkyl,
(c) —C₂₋₆ alkenyl,
(d) hydroxyl,
(e) —O—C₁₋₆ alkyl,
(f) —C₃₋₈ cycloalkyl,
(g) —SO₂—C₁₋₆ alkyl,
(h) —SO₂—NR^{8A}R^{8B},
(i) —C₀₋₆ alkyl-C(═O)—Oₙ—R¹⁰,
(j) —C₂₋₆ alkenyl-C(═O)—Oₙ—R¹⁰,
(k) —CN,
(l) —C₀₋₆ alkyl-NR^{8A}R^{8B},
(m) —C₀₋₆ alkyl-C(═O)—NR^{8A}R^{8B},
(n) —NR^{9A}—C₀₋₆ alkyl-C(═O)—Oₙ—R^{9B},
(o) —C₆₋₁₀ aryl,
(p) heteroaryl, wherein said heteroaryl group has 5 to 12 ring atoms selected from C, N, O and S,
wherein said alkyl or alkenyl moiety is optionally substituted with one or more
(I) halogen,
(II) hydroxyl,
(III) CN, or
(IV) —NR^{9A}R^{9B};
R⁵ and R⁶ are selected from the group consisting of
(1) hydrogen,
(2) —NR^{9A}R^{9B},
(3) —C₁₋₃ alkyl,
or R⁵ and R⁶ are linked together to form a C₃₋₈ cycloalkyl group;
R^{8A} and R^{8B} are each selected from the group consisting of
(1) hydrogen,
(2) —C₁₋₆ alkyl,
wherein said R^{8A} and R^{8B} alkyl moiety is optionally substituted with one or more halogen or NR^{9A}R^{9B},
or R^{8A} and R^{8B} are linked together to form a non-aromatic cyclic ring having from 5 to 12 ring atoms selected from C, N O and S, wherein said cyclic ring is optionally substituted with one or more C₁₋₆ alkyl;
R^{9A} and R^{9B} are each selected from the group consisting of
(1) hydrogen, or
(2) —C₁₋₆ alkyl;
R¹⁰ is selected from the group consisting of
(1) hydrogen, or
(2) —C₁₋₆ alkyl; and
n is 0 or 1.

2. A compound of claim 1, wherein Q¹ is a bond.

3. A compound of claim 1, wherein Q¹ is —NH—.

4. A compound of claim 1, wherein Q¹ is —O—C₁₋₂ alkylene-.

5. A compound of claim 1, wherein Q² is a bond.

6. A compound of claim 1, wherein Q² is —NH—(CH₂)ₙ—.

7. A compound of claim 1, wherein Q² is

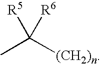

8. A compound of claim 1, wherein R¹ is methyl.

9. A compound of claim 1, wherein R³ is C₆₋₁₀ aryl, which is optionally substituted with one or more
(a) —C₁₋₆ alkyl,
(b) —O—C₁₋₆ alkyl,
(c) —NR^{8A}R^{8B}, or
(d) —CN,
wherein said alkyl moiety is optionally substituted with one or more halogen or CN.

10. A compound of claim 1, wherein R³ is heteroaryl, which is optionally substituted with one or more
(a) —C₁₋₆ alkyl,
(b) —NR^{8A}R^{8B}, or
(c) —CN,
wherein said alkyl moiety is optionally substituted with one or more halogen or CN.

11. A compound of claim 1, wherein R² is C₆₋₁₀ aryl, wherein said aryl group is optionally substituted with one or more
(a) halogen,
(b) —C₁₋₆ alkyl,
(c) hydroxyl,
(d) —O—C₁₋₆ alkyl,
(e) —SO₂—C₁₋₆ alkyl,
(f) —C₀₋₆ alkyl-C(═O)—Oₙ—R¹⁰,
(g) —C₂₋₆ alkenyl—C(═O)—Oₙ—R¹⁰,
(h) —CN,
(i) —C₀₋₆ alkyl-NR^{8A}R^{8B},
(j) —C₀-6 alkyl-C(═O)—NR^{8A}R^{8B},
(k) —NR^{9A}—C₀₋₆ alkyl-C(═O)—Oₙ—R¹⁰,
(l) heteroaryl, wherein said heteroaryl group has 5 to 12 ring atoms selected from C, N, O and S,
wherein said alkyl or alkenyl moiety is optionally substituted with one or more
(I) halogen,
(II) hydroxyl, or
(III) CN.

12. A compound of claim 1, wherein R² is heteroaryl, wherein said heteroaryl group has 5 to 12 ring atoms selected from C, N, O and S, and said heteroaryl is optionally substituted with one or more
(a) halogen,
(b) —C₁₋₆ alkyl,
(c) hydroxyl,
(d) —O—C₁₋₆ alkyl,
(e) —SO₂—C₁₋₆ alkyl,
(f) —C₀₋₆ alkyl-C(═O)—Oₙ—R¹⁰,
(g) —C₂₋₆ alkenyl—C(═O)—Oₙ—R¹⁰,
(h) —CN,
(i) —C₀₋₆ alkyl-NR^{8A}R^{8B},
(j) —C₀₋₆ alkyl-C(═O)—NR^{8A}R^{8B},
(k) —NR^{9A}—C₀₋₆ alkyl-C(═O)—Oₙ—R¹⁰,
(l) heteroaryl, wherein said heteroaryl group has 5 to 12 ring atoms selected from C, N, O and S,
wherein said alkyl or alkenyl moiety is optionally substituted with one or more
(I) halogen,
(II) hydroxyl, or
(III) CN.

13. A compound of claim 1, wherein the compound of formula (I) is a compound of formula (II): 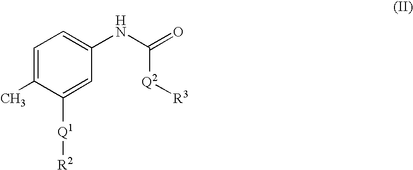
or a pharmaceutically acceptable salt thereof.

14. A compound of claim 1, wherein the compound of formula (I) is a compound of formula (III): 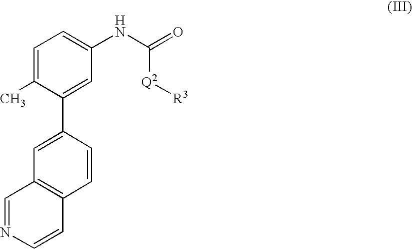
or a pharmaceutically acceptable salt thereof.

15. A compound of claim 1, wherein the compound of formula (I) is a compound of formula (IV): 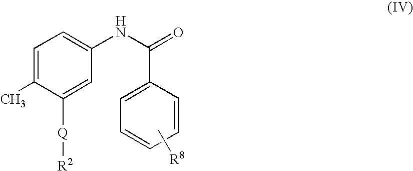
or a pharmaceutically acceptable salt thereof, wherein R⁸ is optionally present at one or more of the phenyl carbon atoms and is selected from the group consisting of
(a) —C₁₋₆ alkyl,
(b) —O—C₁₋₆ alkyl,
(c) —NR^{8A}R^{8B}, or
(d) —CN,
wherein said alkyl moiety is optionally substituted with one or more halogen or CN.

16. A compound of claim 1, which is selected from the group consisting of 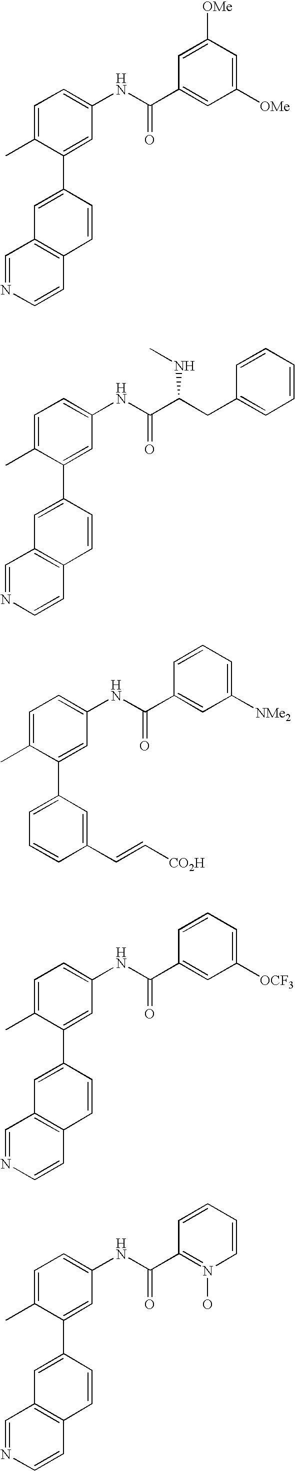 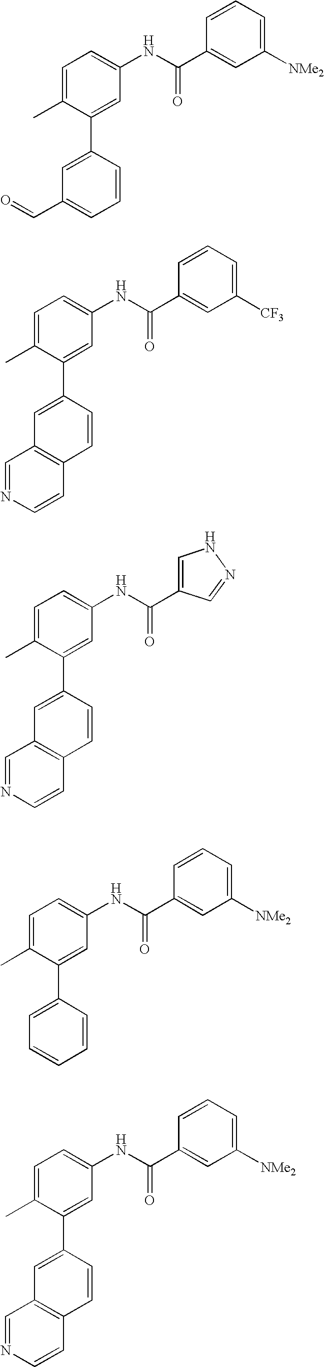 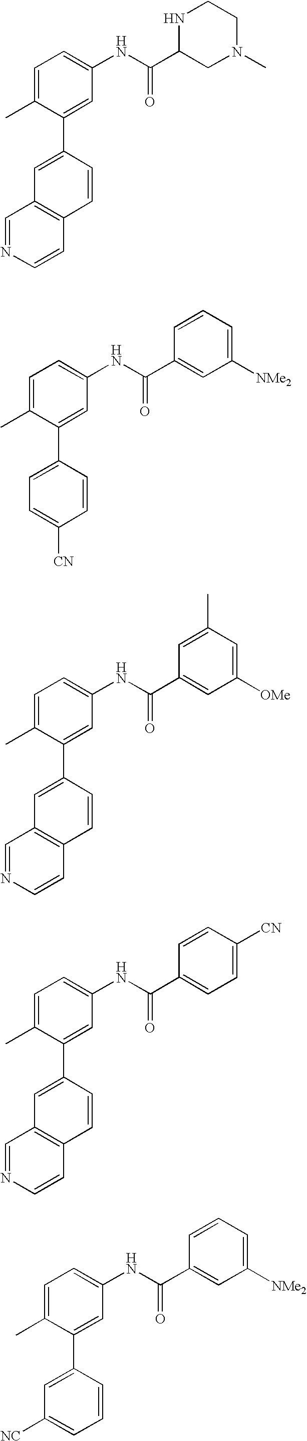 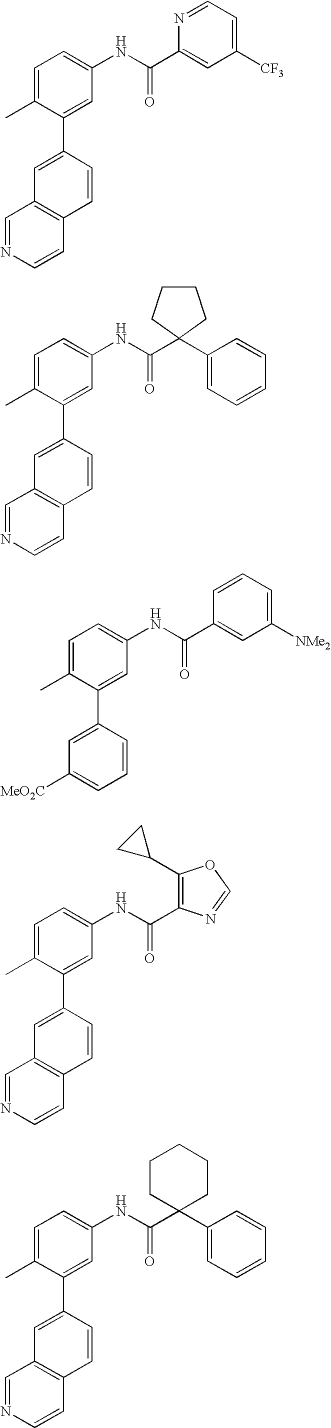 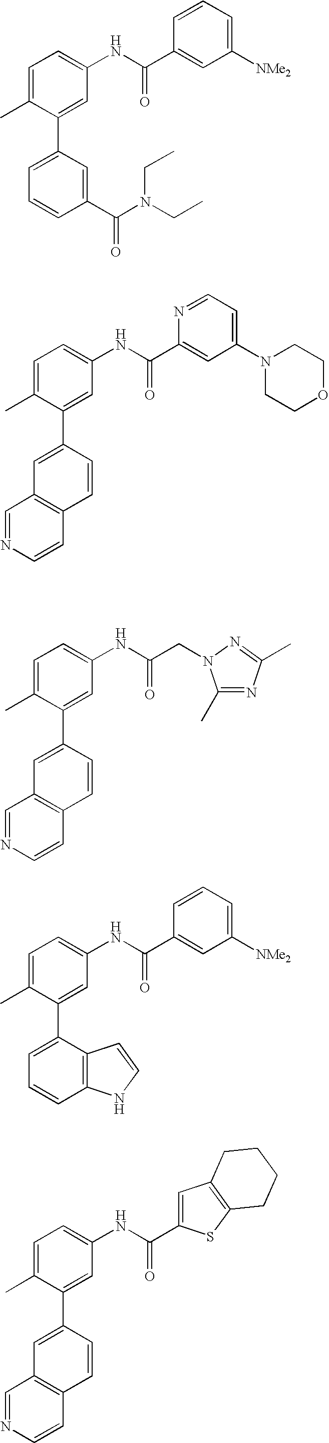 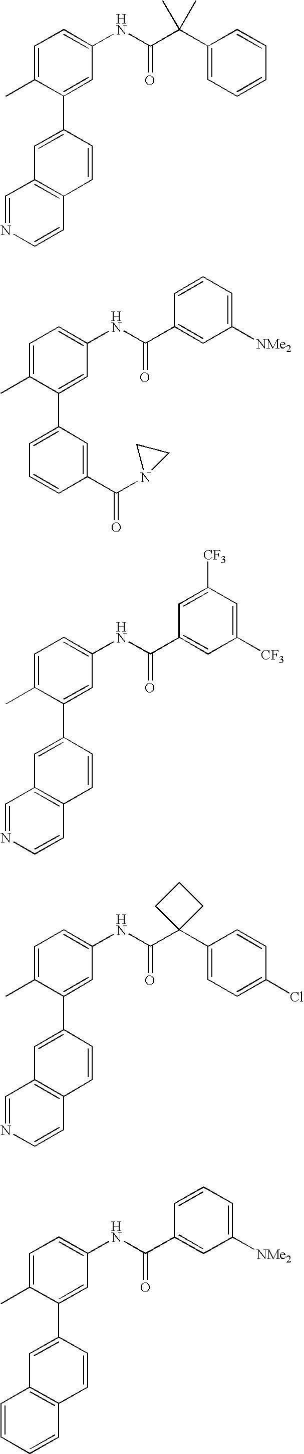 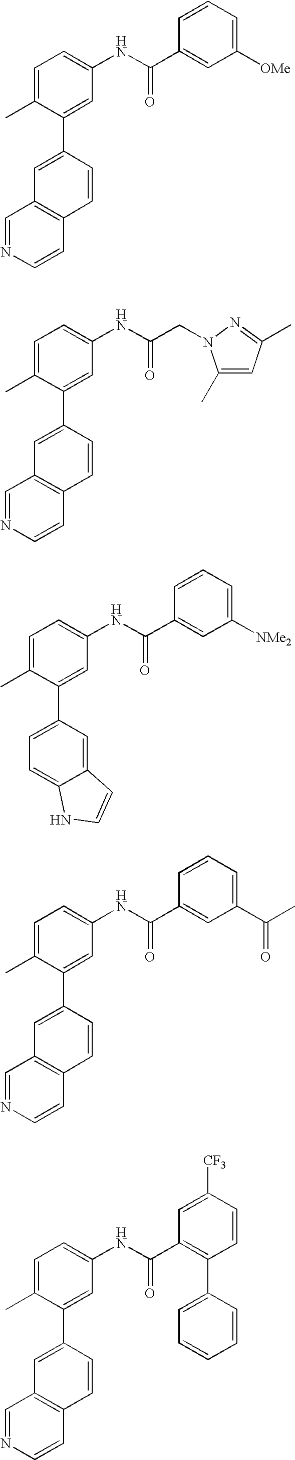 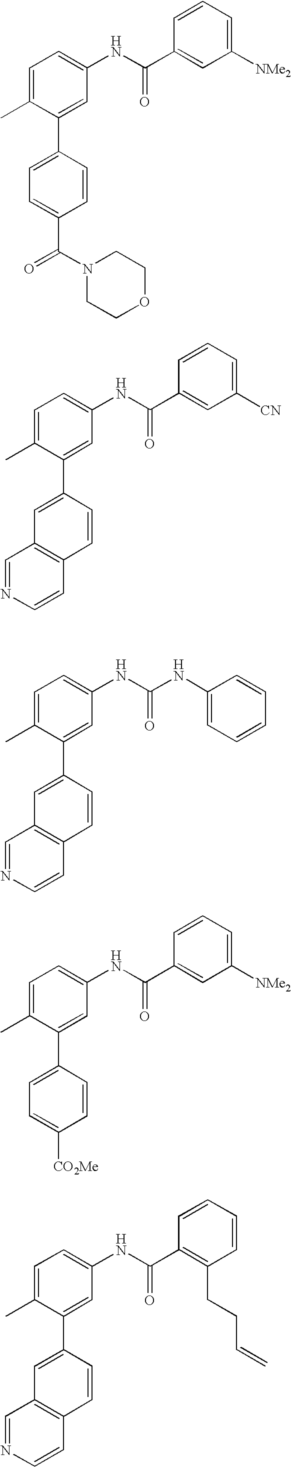 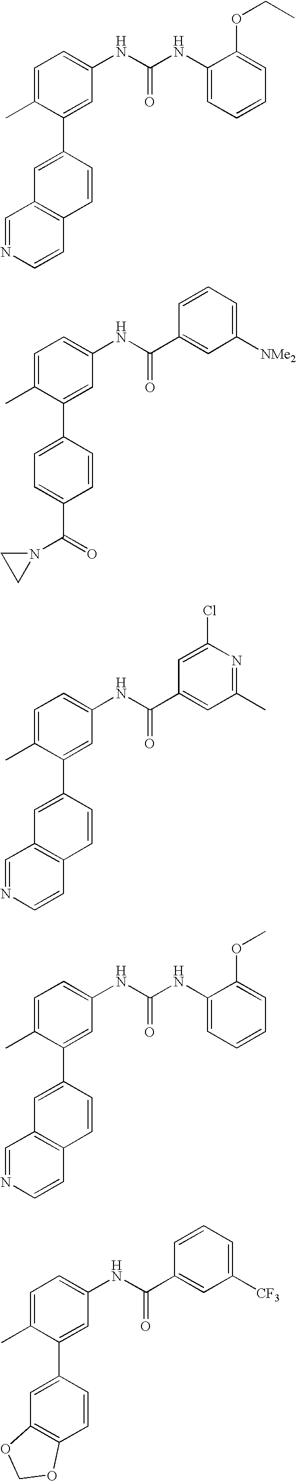 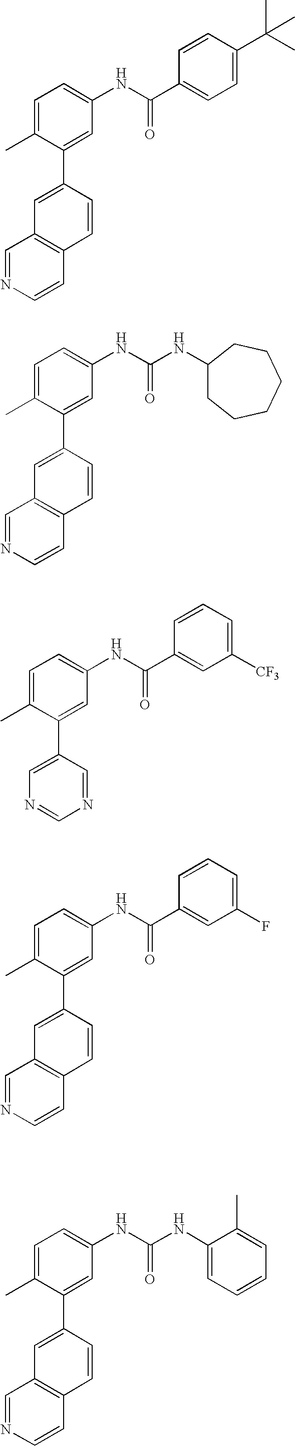 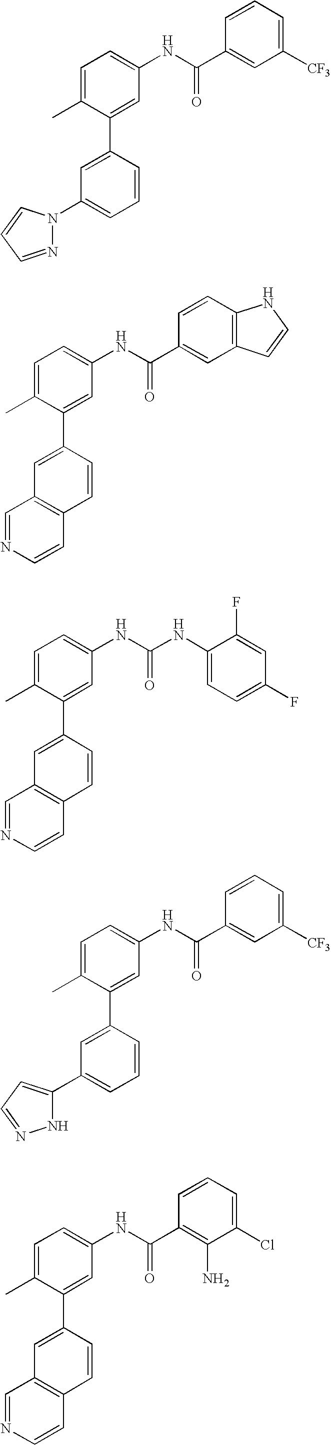 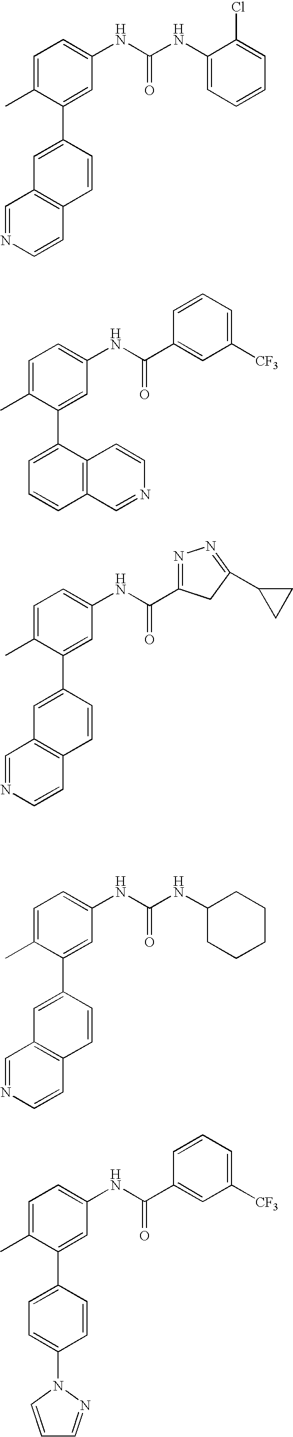 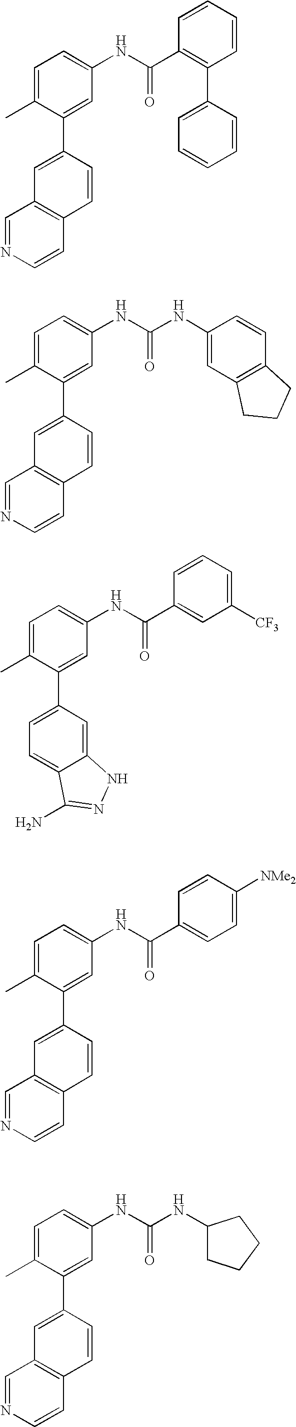 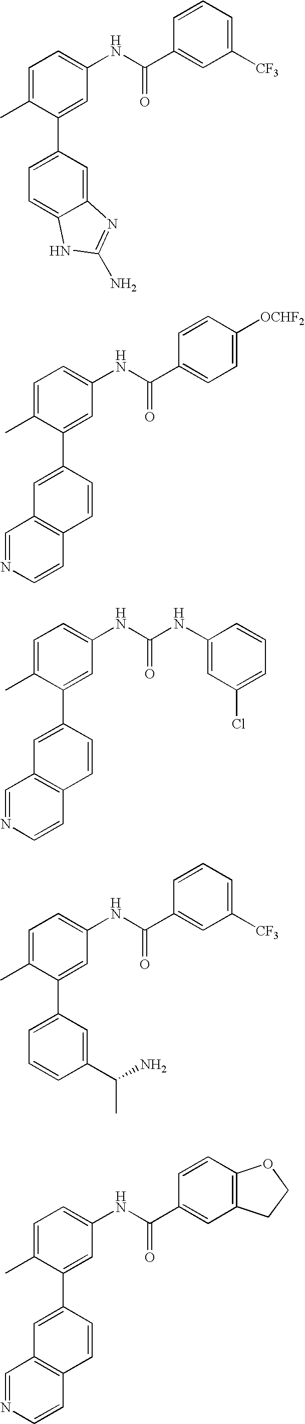 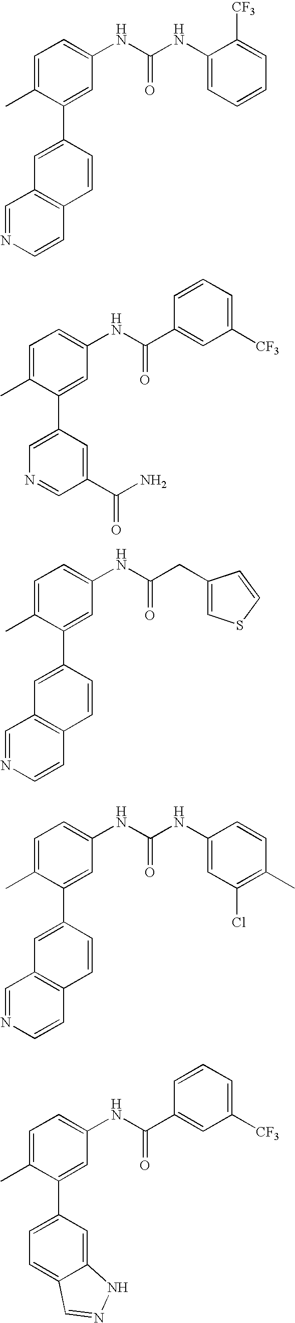 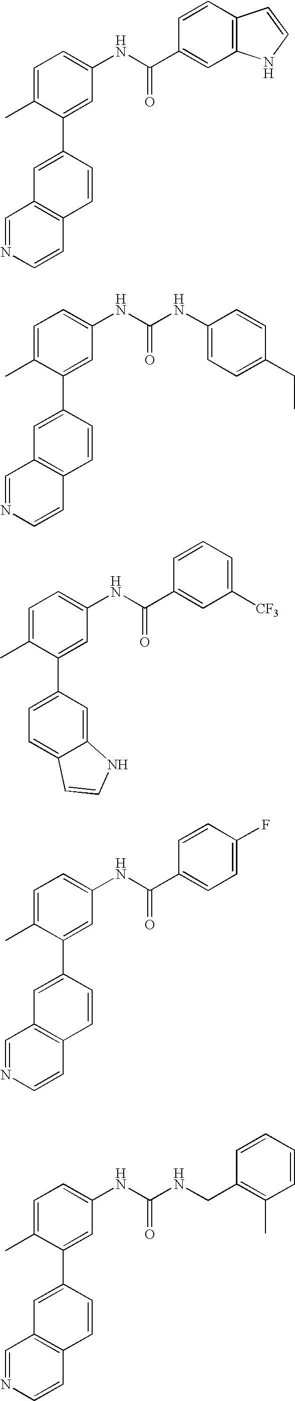 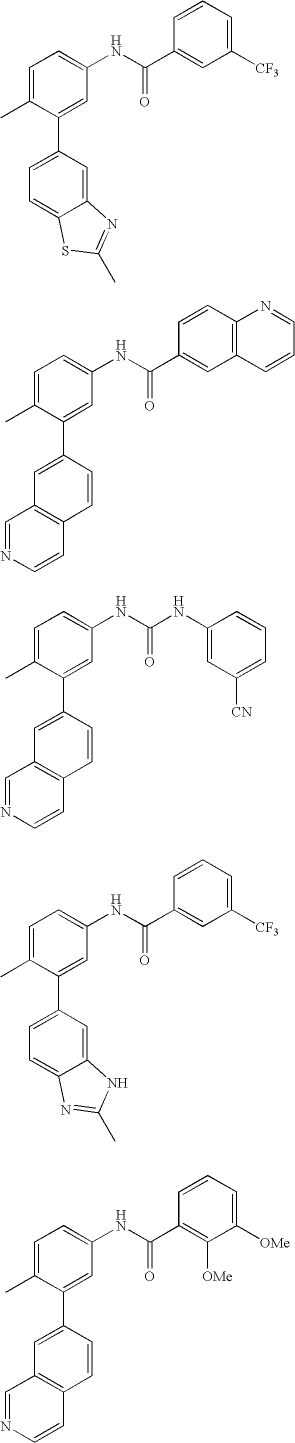 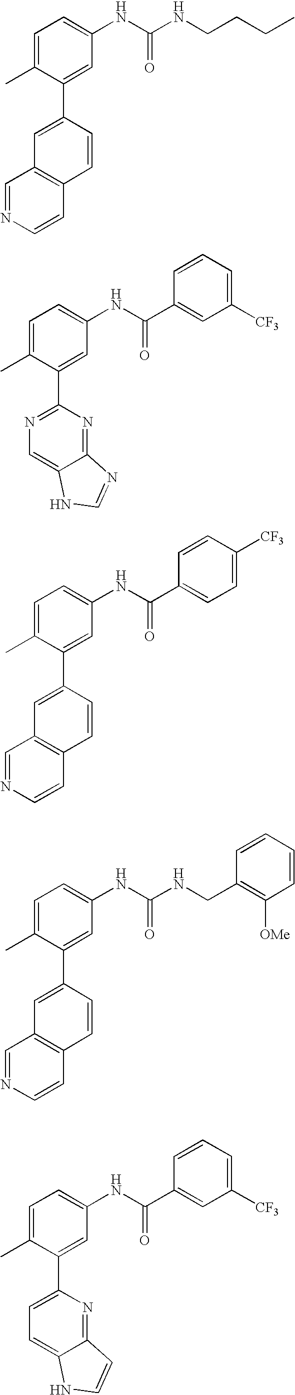 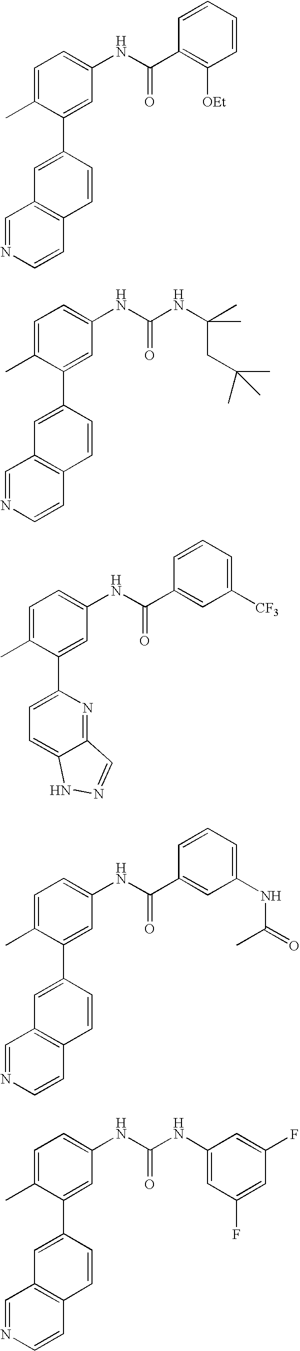 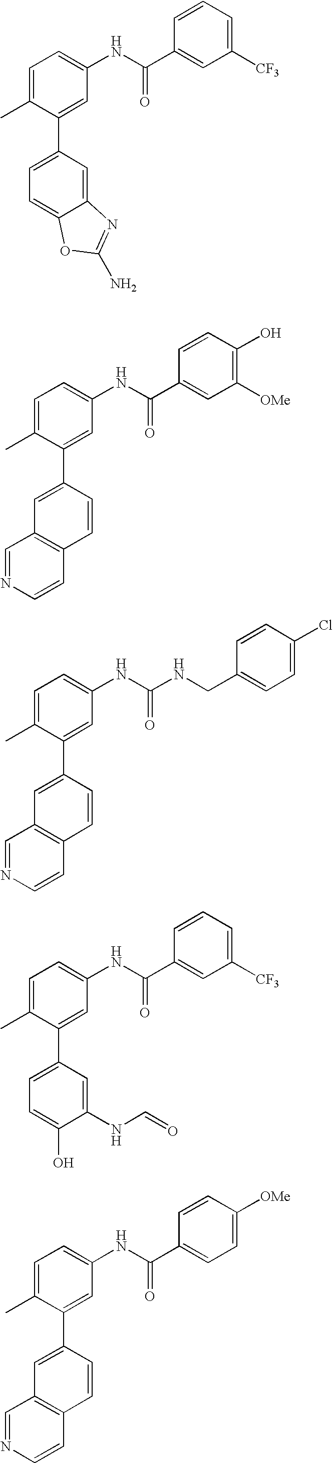 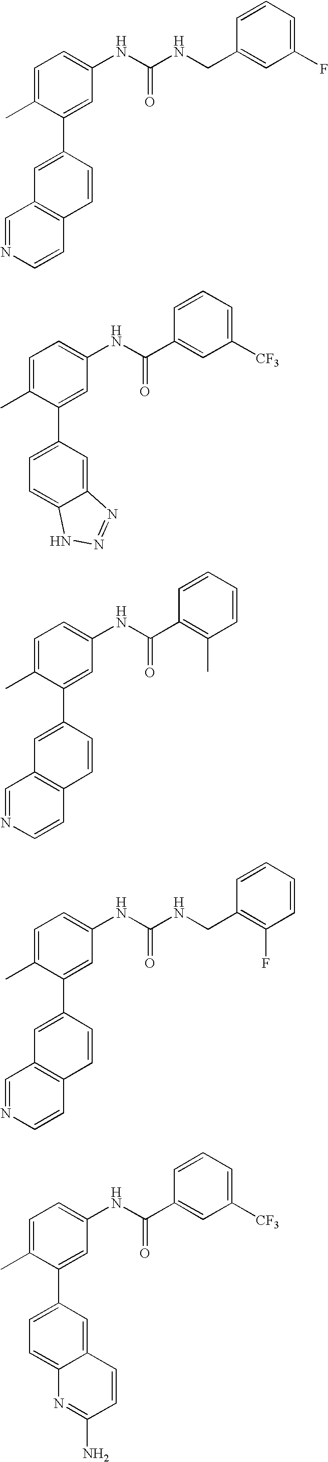 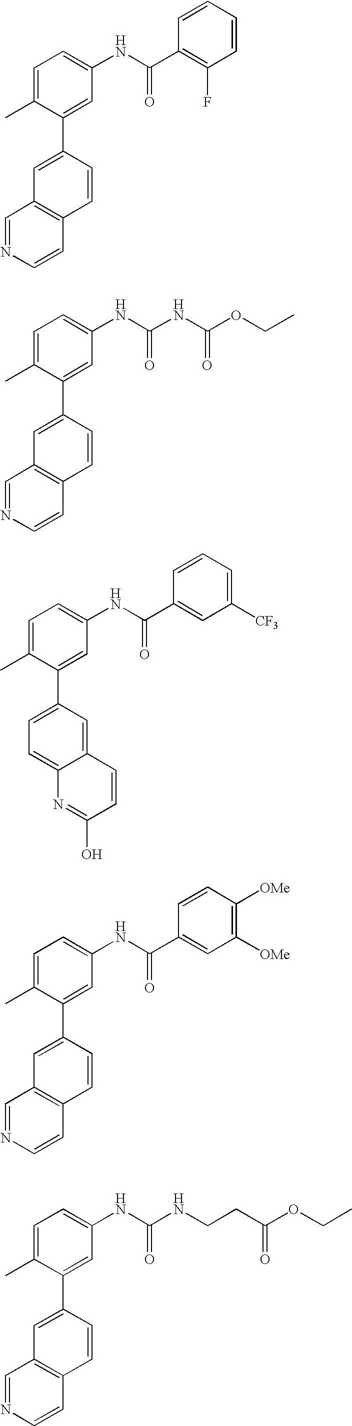 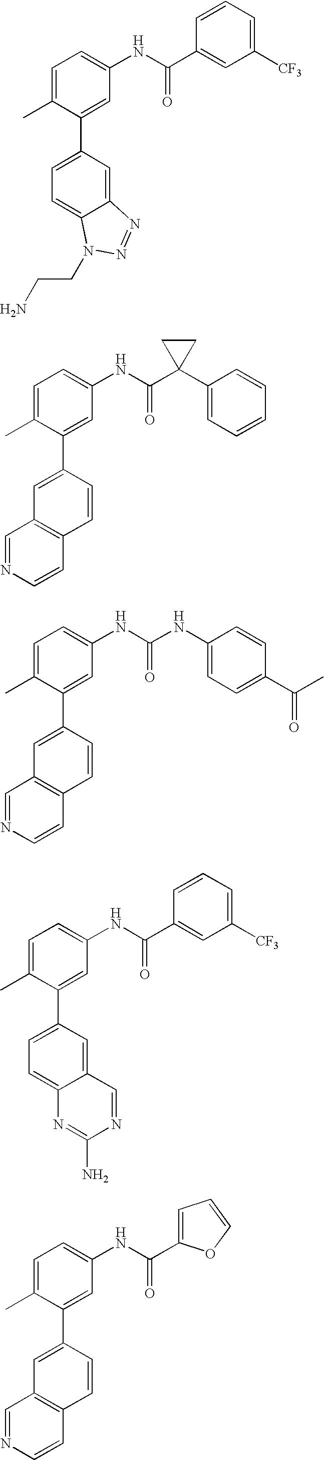 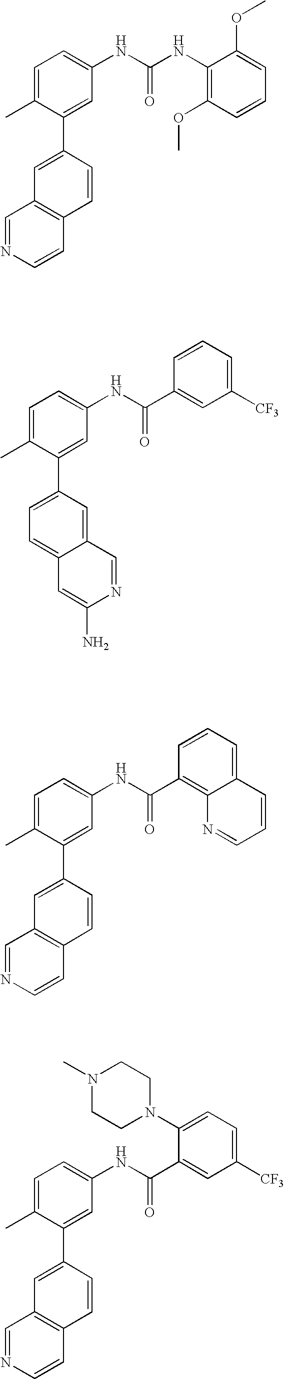 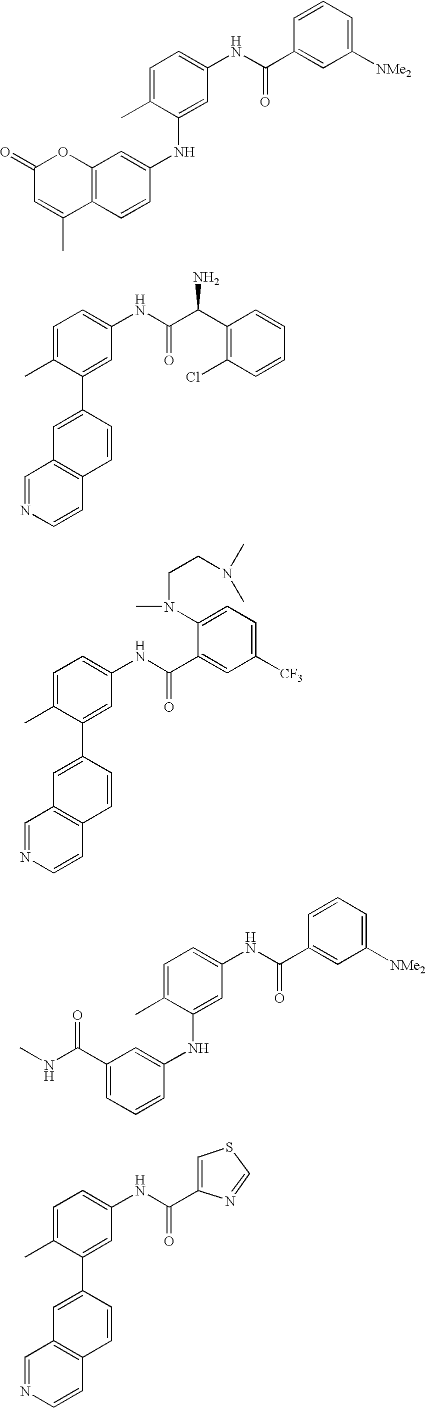 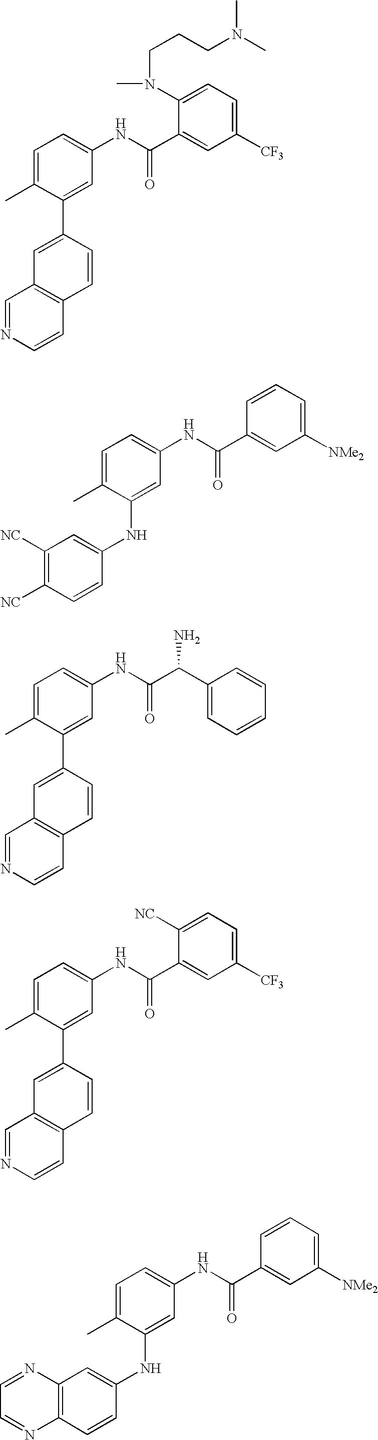 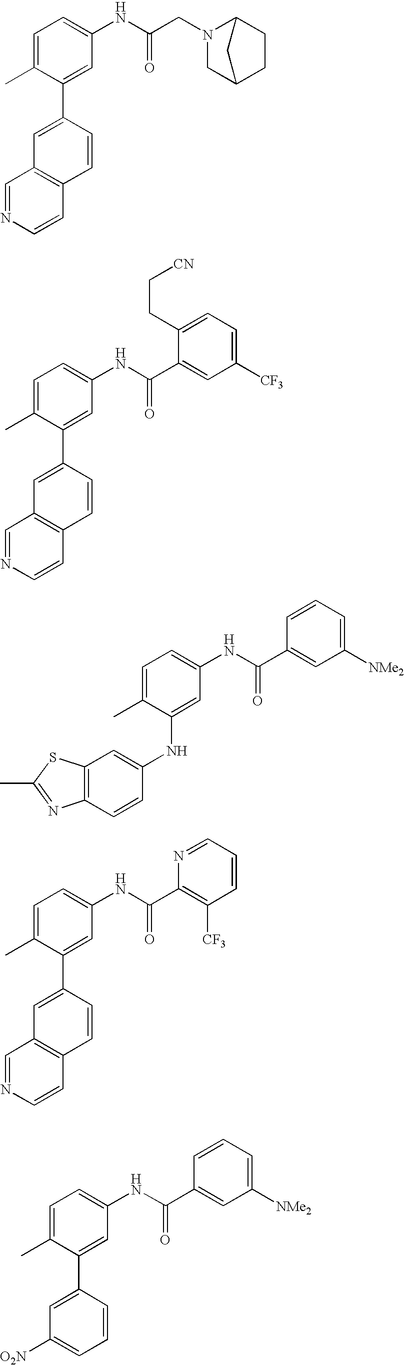 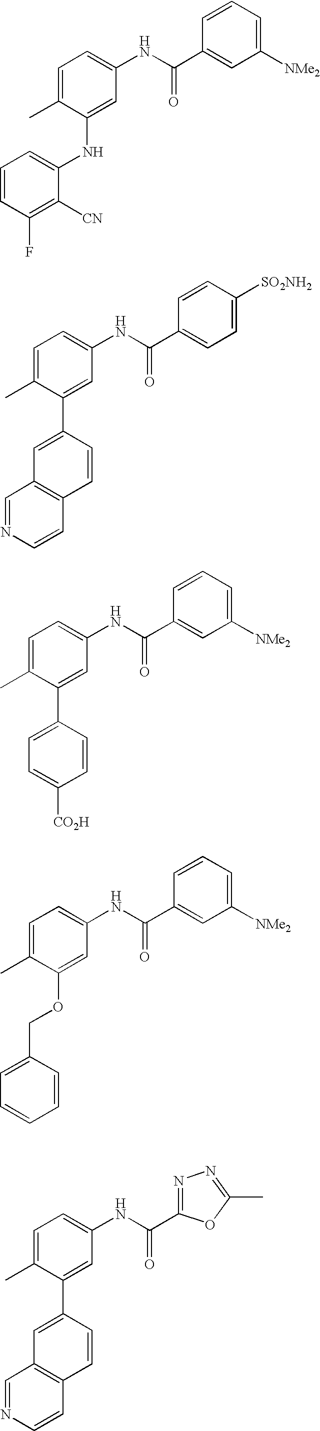 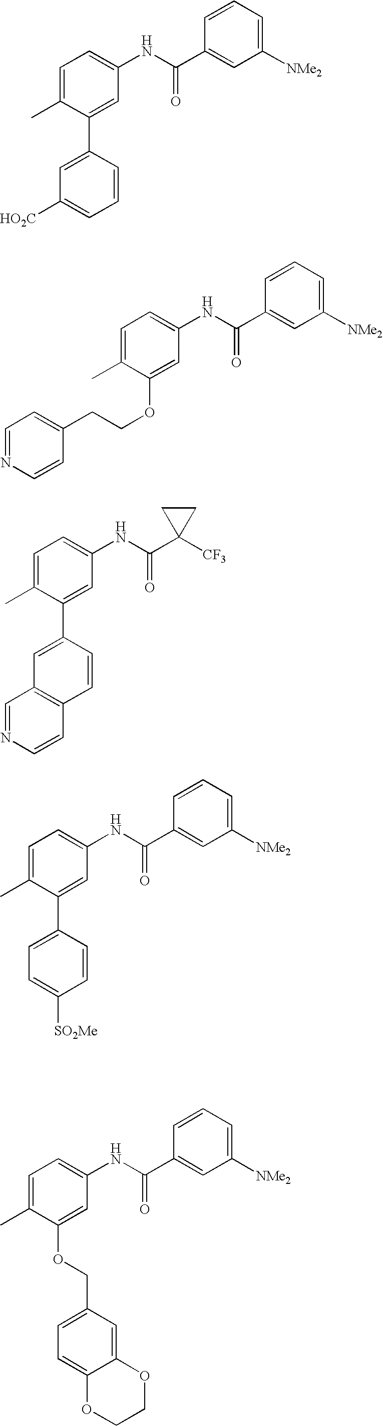 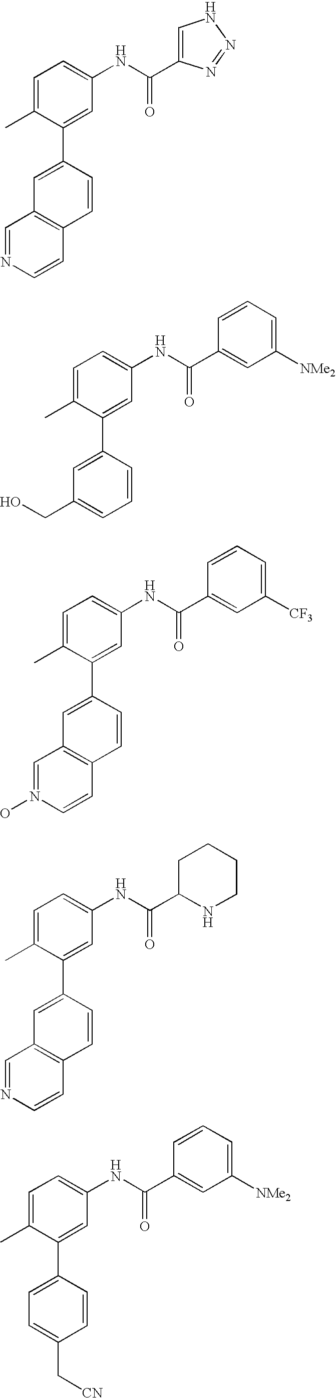
or a pharmaceutically acceptable salt thereof.

17. A pharmaceutical composition comprising a therapeutically effective amount of a compound of claim 1, or a pharmaceutically acceptable salt thereof, and a pharmaceutically acceptable carrier.

18. A method of treating a disease or disorder regulated by EphA4 RTK signaling, wherein said disease or disorder is selected from the group consisting of stroke, spinal cord injury, traumatic brain injury, Alzheimer's Disease, Parkinson's Disease, multiple sclerosis, amyotrophic lateral sclerosis, Huntington's Disease, rheumatoid arthritis, asthma, chronic obstructive pulmonary disease, Crohn's disease, psoriasis, atherosclerosis, diabetic and other retinophathies, age-related macular degeneration, neovascular glaucoma, vascular diseases and cancer, comprising administering to the patient a therapeutically effective amount of a compound of claim 1, or a pharmaceutically acceptable salt thereof, and a pharmaceutically acceptable carrier.
